(19) United States
(12) Patent Application Publication
(10) Pub. No.: US 2024/0100065 A1
(43) Pub. Date: Mar. 28, 2024

(54) METHODS FOR TREATING RECEPTOR-INTERACTING PROTEIN KINASE 1-MEDIATED DISEASES

(71) Applicant: Genzyme Corporation, Cambridge, MA (US)

(72) Inventors: Nazem Atassi, Cambridge, MA (US); Catherine Cantalloube, Paris (FR); Agnès Hincelin-Mery, Paris (FR); Donato Teutonico, Paris (FR)

(73) Assignee: Genzyme Corporation, Cambridge, MA (US)

(21) Appl. No.: 18/236,462

(22) Filed: Aug. 22, 2023

Related U.S. Application Data
(60) Provisional application No. 63/400,384, filed on Aug. 23, 2022.
(60) Provisional application No. 63/420,932, filed on Oct. 31, 2022.

(51) Int. Cl. A61K 31/553; A61K 9/00

(52) U.S. Cl. CPC A61K 31/553; A61K 9/0056

(57) ABSTRACT

This disclosure relates to the field of therapeutic tyrosine kinase inhibitors, in particular receptor-interacting serine/threonine-protein kinase 1 (RIPK1) inhibitors, to treat a receptor-interacting protein kinase 1-mediated disease or disorder.

### CROSS-REFERENCE TO RELATED APPLICATIONS

This application claims priority to U.S. Provisional Application No. 63/400,384, filed on Aug. 23, 2022, and U.S. Provisional Application No. 63/420,932, filed on Oct. 31, 2022, which are incorporated by reference herein in their entirety for any purpose.

### FIELD OF THE DISCLOSURE

The present disclosure relates to the field of therapeutic kinase inhibitors, in particular receptor-interacting serine/threonine-protein kinase 1 (RIPK1) inhibitors, to treat receptor-interacting protein kinase 1-mediated disease.

### BACKGROUND

RIPK1 is an intracellular protein involved in the regulation of inflammation, cytokine release, and cell death. RIPK1 is activated in response to several inflammatory stimuli, most notably tumor necrosis factor alpha (TNF-α) signaling through its receptor 1 (TNF1), with subsequent RIPK1 initiation of a complex signaling cascade that triggers intracellular responses, including cytokine release, microglial activation, and necroptosis, a regulated form of cell death. Inhibition of RIPK1 activity has been shown to protect against necroptotic cell death in vitro across a range of cell death models. In the central nervous system (CNS), RIPK1 is expressed in all major cell types. The kinase activity of RIPK1 regulates both caspase-independent necroptotic cell death via RIPK3 and MLKL and RIPK1-dependent apoptosis via caspase-8, and emerging evidence suggests RIPK1 can also regulate pro-inflammatory cytokine and chemokine production. These multiple consequences of RIPK1 signaling have raised the possibility that a CNS-penetrant inhibitor of RIPK1 could be beneficial in preventing inflammation and necroptosis in chronic CNS diseases in humans. In various animal models, inhibition of RIPK1 protects against their respective pathologies and attributed cell death. These nonclinical findings, coupled with observations of increased RIPK1 activity in human diseases, suggest that inhibition of RIPK1 can be effective therapies for diseases involving inflammation or cell death.

### BRIEF DESCRIPTION

In one embodiment, the present disclosure provides a method of treating a receptor-interacting protein kinase 1-mediated disease or disorder, comprising administering to a subject in need thereof a therapeutically effective amount of 4-(3,3-difluoro-2,2-dimethyl-propanoyl)-3,5-dihydro-2H-pyrido[3,4-f] [1,4]oxazepine-9-carbonitrile, or a pharmaceutically acceptable salt thereof.

In one embodiment, the present disclosure provides a method of treating a receptor-interacting protein kinase 1-mediated disease or disorder, comprising administering to a subject in need thereof at a dose of about 5 to 60 mg (measured as the equivalent amount of free base) per day of 4-(3,3-difluoro-2,2-dimethyl-propanoyl)-3,5-dihydro-2H-pyrido[3,4-f] [1,4]oxazepine-9-carbonitrile, or a pharmaceutically acceptable salt thereof.

In one embodiment, the present disclosure provides 4-(3,3-difluoro-2,2-dimethyl-propanoyl)-3,5-dihydro-2H-pyrido[3,4-f] [1,4]oxazepine-9-carbonitrile, or a pharmaceutically acceptable salt thereof is used in the treatment of a receptor-interacting protein kinase 1-mediated disease or disorder at a dose of about 5 to 60 mg (measured as the equivalent amount of free base) per day.

In one embodiment, the present disclosure provides an article of manufacture, a packaging, or an administration unit, comprising:
a packaging material;
the above defined pharmaceutical composition; and
a label or package insert contained within said packaging material, indicating that said pharmaceutical composition is administered to a patient for the treatment of a receptor-interacting protein kinase 1-mediated disease or disorder, at a dose of about 5 to 60 mg per day of 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof.

In one embodiment, the present disclosure provides a dose of about 5 to 60 mg (measured as the equivalent amount of free base) per day of 4-(3,3-difluoro-2,2-dimethyl-propanoyl)-3,5-dihydro-2H-pyrido[3,4-f] [1,4]oxazepine-9-carbonitrile, or a pharmaceutically acceptable salt thereof, for the manufacture of a medicament useful in a receptor-interacting protein kinase 1-mediated disease or disorder. In another embodiment, herein is provided a dose of about 5 to 60 mg of compound (1), or a pharmaceutically acceptable salt thereof, for the manufacture of a medicament useful in treating a receptor-interacting protein kinase 1-mediated disease or disorder.

### BRIEF DESCRIPTION OF FIGURES

FIG. 1: Line plot of median (Q1-Q3) percent change from baseline in pRIPK1 over the course of the study by dose level group—Pharmacodynamic population. B=Baseline (done on Day−1 or Day1 prior to dosing). Cohorts 1, 2, and 4: For a given timepoint, the mean of subtracted values of 2 replicates is plotted for each participant when the coefficient of variation of the 2 values is <=20%. When the value of the 2^{nd} replicate is missing (replicate declared ‘QNS’), the value of the 1^{st} replicate is considered (if the 1^{st} replicate is not declared ‘QNS’). Cohort 3: For a given timepoint, only the subtracted value of the 1^{st} replicate is plotted for each participant due to a database issue.

FIG. 2: Line plot of median (Q1-Q3) percent change from baseline in pRIPK1 over the course of the study by dose level group—Pharmacodynamic population. B=Baseline (done on Day−1 or Day1 prior to dosing). The mean of blank-subtracted values was taken into account in the statistical analysis when CV %<=20% between both replicates and the bioanalysis considered bother replicates correct. Otherwise, the value of only one replicate was taken into account upon bioanalyst consideration. Else, bioanalysis of both replicates was considered as failed (missing value). Reference line: 90% of inhibition.

FIG. 3: 3A: Observed and predicted plasma concentration of Compound 1 after single dose of 10 mg-part 1b (black line represents the median, grey area the 90% PI); 3B: Observed and predicted plasma concentration of Compound 1 after single dose of 40 mg-part 1b; 3C: Observed and predicted CSF concentration of Compound 1 after single dose of 10 mg and 40 mg-part 1b; 3D: Predicted plasma and CSF concentration after repeated administration of 20 mg QD (median curve and area the 90% predicted interval); 3E: Predicted plasma and CSF concentration after repeated administration of 20 mg BID (median curve and area the 90% predicted interval).

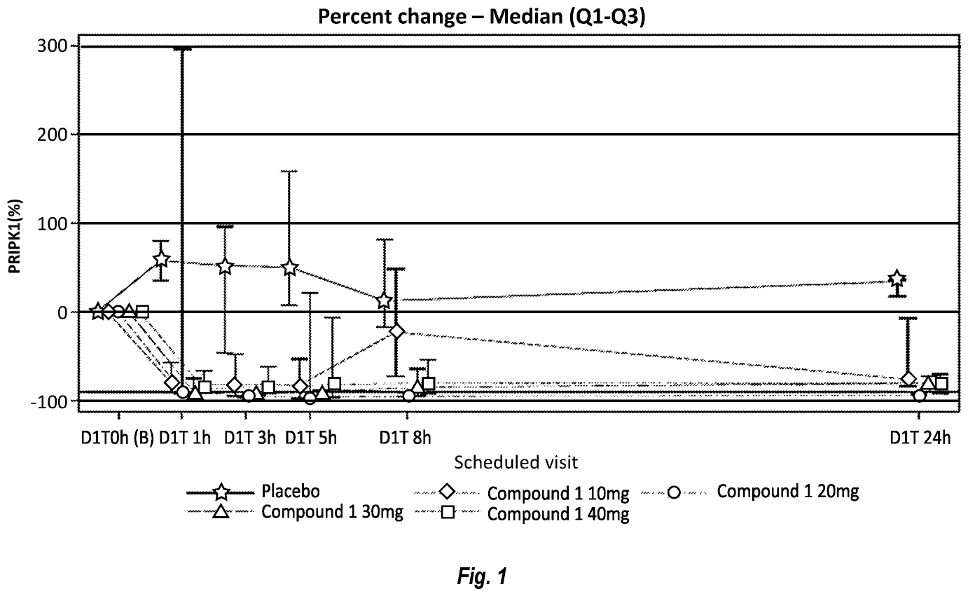

### DETAILED DESCRIPTION

While the disclosure provides illustrated embodiments, it will be understood that they are not intended to limit the disclosure to those embodiments. On the contrary, the disclosure is intended to cover all alternatives, modifications, and equivalents, which may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art. The present disclosure includes, for example, any one or a combination of the following embodiments.

### Definitions

Unless otherwise stated, the following terms used in the specification and claims are defined for the purposes of this disclosure and have the following meaning:

As used herein, “the RIPK1 inhibitor,” “the RIPK1 inhibitor compound,” “Compound 1,” and “the compound”, refers to 4-(3,3-difluoro-2,2-dimethyl-propanoyl)-3,5-dihydro-2H-pyrido[3,4-f] [1,4]oxazepine-9-carbonitrile having the following structure:

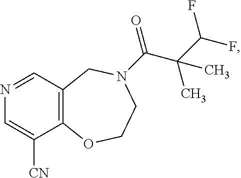 [SMILES: CC(C)(C(=O)N1CCOc2c(C#N)cncc2C1)C(F)F]

and/or a pharmaceutically acceptable salt thereof.

As used herein, the term “free base” means the compound referenced per se, e.g. not in the form of a salt or prodrug. Reference to, e.g., a “dose of . . . 5 mg” means that there is an amount of compound present, whether in the form of the free base or a salt or prodrug, which corresponds to the same molar amount of 5 mg of the freebase, i.e. the compound in salt form would have a mass of greater than 5 mg. Unless otherwise indicated, an amount of an active compound for administration (whether administered as a free base or as a salt form) refers to or is based on the amount of the compound in free base form.

The phrase “pharmaceutically acceptable” is employed herein to refer to those compounds, materials, compositions and/or dosage forms which are, within the scope of sound medical judgment, suitable for use in contact with the tissues of human beings without excessive toxicity, irritation, allergic response or other problem or complication, commensurate with a reasonable benefit/risk ratio.

As used herein, the term “pharmaceutically acceptable salt” refers to the relatively non-toxic, inorganic and organic acid addition salts, and base addition salts, of a compound. These salts can be prepared in situ during the final isolation and purification of the compounds.

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) preventing the disease, e.g., causing the clinical symptoms of the disease not to develop in a mammal that may be exposed to or predisposed to the disease but does not yet experience or display symptoms of the disease;
(2) inhibiting the disease, e.g., arresting or reducing the development of the disease or its clinical symptoms; inhibiting further progression or worsening of at least one symptom, i.e. by reducing the severity or frequency of at least one symptom.
(3) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the RIPK1 inhibitor compound, that, when administered to a mammal for treating a disease, is sufficient to effect such treatment for the disease. The “therapeutically effective amount” will vary depending on the compound, the disease and its severity and the age, weight, etc., of the mammal to be treated.

Before describing the present teachings in detail, it is to be understood that the disclosure is not limited to specific compositions or process steps, as such may vary.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Unless specifically stated or obvious from the context, as used herein, the term “about” refers to a value that is within an acceptable error range for a particular value determined by a person of ordinary skill, a portion of which will depend on how the measurement or determination is made. For example, “about” may mean a range of up to 10% (ie, ±10%). Therefore, “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “about” should be assumed to be within an acceptable error range for that specific value.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

Unless specifically noted in the above specification, embodiments in the specification that recite “dose” weight described herein refers to the weight of free base of a compound (active moiety).

“Or” is used in the inclusive sense, i.e., equivalent to “and/or,” unless the context requires otherwise.

As used herein, the term “QD” or “OD” refers to every day, once daily, once a day, per day; “BID” refers to twice a day.

### Administered RIPK1 Inhibitor Compound and Pharmaceutical Compositions

In some embodiments, a RIPK1 inhibitor compound, 4-(3,3-difluoro-2,2-dimethyl-propanoyl)-3,5-dihydro-2H-pyrido[3,4-f] [1,4]oxazepine-9-carbonitrile is administered for treating a receptor-interacting protein kinase 1-mediated disease or disorder in a subject in need thereof. In some embodiments, the RIPK1 inhibitor compound is a pharmaceutically acceptable salt of 4-(3,3-difluoro-2,2-dimethyl-propanoyl)-3,5-dihydro-2H-pyrido[3,4-f] [1,4]oxazepine-9-carbonitrile. In some embodiments, a therapeutically effective amount of the RIPK1 inhibitor compound is administered. In some embodiments, a dose of 5 to 60 mg of the RIPK1 inhibitor compound is administered.

The RIPK1 inhibitor compound can be prepared according to the methods and schemes described in, e.g., U.S. Pat. No. 11,203,600, in particular Method I, set forth at columns 63-66, which is incorporated herein by reference.

### Pharmaceutical Compositions

Compounds provided herein are usually administered in the form of pharmaceutical compositions. Thus, provided herein are also pharmaceutical compositions that contain one or more of the compounds described herein or a pharmaceutically acceptable salt, tautomer, stereoisomer, mixture of stereoisomers, prodrug, or deuterated analog thereof and one or more pharmaceutically acceptable excipients. Such compositions are prepared in a manner well known in the pharmaceutical arts.

The pharmaceutical compositions are comprised of in general, the RIPK1 inhibitor compound and/or a pharmaceutically acceptable salt thereof in combination with a pharmaceutically acceptable excipient such as binders, surfactants, diluents, buffering agents, antiadherents, glidants, hydrophilic or hydrophobic polymers, retardants, stabilizing agents or stabilizers, disintegrants or superdisintegrants, antioxidants, antifoaming agents, fillers, flavors, colors, lubricants, sorbents, preservatives, plasticizers, or sweeteners, or mixtures thereof, which facilitate processing of the RIPK1 inhibitor compound and/or a pharmaceutically acceptable salt thereof into preparations which can be used pharmaceutically. Any of the well-known techniques and excipients may be used as suitable and as understood in the art, see for example, Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012).

In solid dosage forms of the disclosure for oral administration (capsules, tablets, pills, dragees, powders, granules and the like), the active ingredient is mixed with one or more pharmaceutically-acceptable excipients, such as sodium citrate or dicalcium phosphate and/or any of the following: fillers or extenders, such as starches, lactose, sucrose, glucose, mannitol and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates and sodium carbonate; solution retarding agents, such as paraffin; absorption accelerators, such as quaternary ammonium compounds; wetting agents, such as, for example, acetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like. Example of such formulation include but are not limited to powder in hard gelatin.

### Therapeutic Methods

Provided herein are methods of treating a receptor-interacting protein kinase 1-mediated disease or disorder comprising administering to a subject in need thereof a therapeutically effective amount of the RIPK1 inhibitor compound comprising 4-(3,3-difluoro-2,2-dimethyl-propanoyl)-3,5-dihydro-2H-pyrido[3,4-f] [1,4]oxazepine-9-carbonitrile, and/or a pharmaceutically acceptable salt thereof. Provided herein are methods of treating a receptor-interacting protein kinase 1-mediated disease or disorder comprising administering to a subject in need thereof a therapeutically effective amount of the RIPK1 inhibitor compound comprising 4-(3,3-difluoro-2,2-dimethyl-propanoyl)-3,5-dihydro-2H-pyrido[3,4-f] [1,4]oxazepine-9-carbonitrile. In some embodiments the therapeutically effective amount is about 5 to about 60 mg (measured as the equivalent amount of free base).

In certain embodiments, the disease or disorder is necrotizing enterocolitis, tuberous sclerosis, Tangier's Disease, Wohlman's Syndrome, inflammatory bowel disease, Crohn's disease, ulcerative colitis, psoriasis, retinal detachment, retinitis pigmentosa, macular degeneration, pancreatitis (e.g., acute pancreatitis), atopic dermatitis, rheumatoid arthritis, spondylarthritis, gout, SoJIA, systemic lupus erythematosus, Sjogren's syndrome, systemic scleroderma, anti-phospholipid syndrome, vasculitis, osteoarthritis, non-alcohol steatohepatitis, alcohol steatohepatitis, autoimmune hepatitis autoimmune hepatobiliary diseases, primary sclerosing cholangitis, nephritis, Celiac disease, autoimmune ITP, transplant rejection, ischemia reperfusion injury of solid organs, sepsis, systemic inflammatory response syndrome, cerebrovascular accident, myocardial infarction, Huntington's disease, Parkinson's disease, allergic diseases, asthma, atopic dermatitis, multiple sclerosis, type I diabetes, Wegener's granulomatosis, pulmonary sarcoidosis, Behcet's disease, interleukin-1 converting enzyme associated fever syndrome, chronic obstructive pulmonary disease, tumor necrosis factor receptor-associated periodic syndrome, periodontitis, bacterial infection, staphylococcus infection, mycobacterium infection, retinitis pigmentosa, influenza, transplant rejection, burns or hypoxia. In certain embodiments, the disease or disorder is trauma, ischemia, stroke, cardiac infarction, infection, lysosomal storage disease, Niemann-Pick disease, Gaucher's disease, Krabbe disease, sepsis, Parkinson's disease, amyotrophic lateral sclerosis (ALS/Lou Gehrig's Disease), Huntington's disease, HIV-associated dementia, encephalopathy, retinal degenerative disease, glaucoma, age-related macular degeneration, rheumatoid arthritis, psoriasis, psoriatic arthritis or inflammatory bowel disease. In certain embodiments, the disease or disorder is ALS, Friedreich's ataxia, Huntington's disease, Lewy body disease, Parkinson's disease, Huntington's disease, multiple sclerosis, diabetic neuropathy, polyglutamine (polyQ) diseases, stroke, Fahr disease, Menke's disease, Wilson's disease, cerebral ischemia, lysosomal storage disease or a prion disorder. In certain embodiments, the disease is ALS. In certain embodiments, the disease is. In certain embodiments, the disease is lysosomal storage disease. In certain embodiments, the disease is Parkinson's disease. In certain embodiments the disorder is an ischemic disease of organs including but not limited to brain, heart, kidney and liver. In some different embodiments, the disorder is an ocular disorder such as retinal degenerative disease, glaucoma or age-related macular degeneration. In some different embodiments, the disorder is a central nervous system (CNS) disorder.

In some embodiments, the receptor-interacting protein kinase 1-mediated disease or disorder is trauma, ischemia, stroke, cardiac infarction, infection, Gaucher's disease, Krabbe disease, sepsis, Parkinson's disease, amyotrophic lateral sclerosis, Huntington's disease, HIV-associated dementia, retinal degenerative disease, glaucoma, age-related macular degeneration, rheumatoid arthritis, psoriasis, psoriatic arthritis, or inflammatory bowel disease.

In some embodiments, the receptor-interacting protein kinase 1-mediated disease or disorder is neurodegenerative diseases. Non-limiting examples of neurodegenerative diseases include, amyotrophic lateral sclerosis (ALS), Friedreich's ataxia, Huntington's disease, Lewy body disease, multiple sclerosis, Parkinson's disease, multiple sclerosis, and spinal muscular atrophy.

In certain embodiments, neurodegenerative diseases and CNS diseases include Niemann-Pick disease, type C1 (NPC1), amyotrophic lateral sclerosis (ALS), Friedreich's ataxia, Huntington's disease, Lewy body disease, multiple sclerosis, Parkinson's disease, and spinal muscular atrophy.

In certain embodiments, Compound 1 may be used to treat NPC1 via inhibiting necroptosis that causes neuronal loss.

In certain embodiments, the compound and compositions of the present disclosure are useful for treating Parkinson's disease.

In certain embodiments, the compound and compositions of the present disclosure are useful for treating amyotrophic lateral sclerosis (ALS).

In some embodiments, the subject has one or more symptoms of ALS prior to treatment and the treatment reduces or eliminates the one or more symptoms.

In some embodiments, the subject suffers from neuropathic pain, musculoskeletal pain, or spasticity caused by ALS.

In some embodiments, a subject with ALS has at least one documented relapse within the previous year, and/or greater than two documented relapses within the previous two years, and/or greater than one active Gd-enhancing brain lesion on an MRI scan in the past six months and prior to screening.

In certain embodiments, the compound and compositions of the present disclosure are useful for treating multiple sclerosis.

In one embodiment, the present disclosure provides a method of treating a receptor-interacting protein kinase 1-mediated disease or disorder, comprising administering to a subject in need thereof a therapeutically effective amount of Compound 1, or a pharmaceutically acceptable salt thereof.

In one embodiment, the present disclosure provides a method of treating a receptor-interacting protein kinase 1-mediated disease or disorder, comprising administering to a subject in need thereof Compound 1, or a pharmaceutically acceptable salt thereof; wherein the compound is administered at a dose of about 5 to 60 mg (measured as the equivalent amount of free base) per day.

In one embodiment, the present disclosure provides a method of treating a neurodegenerative disease, comprising administering to a subject in need thereof a dose of about 5 to 60 mg (measured as the equivalent amount of free base) per day of Compound 1, or a pharmaceutically acceptable salt thereof.

In one embodiment, the present disclosure provides a method of treating a neurodegenerative disease, comprising administering to a subject in need thereof a dose of about 5 to 60 mg (measured as the equivalent amount of free base) per day of Compound 1.

In one embodiment, the present disclosure provides a method of treating amyotrophic lateral sclerosis, comprising administering to a subject in need thereof a dose of about 5 to 60 mg per day Compound 1, or a pharmaceutically acceptable salt thereof.

In one embodiment, the present disclosure provides a method of treating a multiple sclerosis, comprising administering to a subject in need thereof a dose of about 5 to 60 mg per day Compound 1, or a pharmaceutically acceptable salt thereof.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg is administered (measured as the equivalent amount of free base). In some embodiments, the dose is about 5 mg, about 10 mg, about 15 mg, about 20 mg, about 25 mg, about 30 mg, about 35 mg, about 40 mg, about 45 mg, about 50 mg, about 55 mg, or about 60 mg (measured as the equivalent amount of free base). In some embodiments, the dose is about 5 mg. In some embodiments, the dose is about 10 mg. In some embodiments, the dose is about 15 mg. In some embodiments, the dose is about 20 mg. In some embodiments, the dose is about 30 mg. In some embodiments, the dose is about 40 mg. In some embodiments, the dose is about 60 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily. In some embodiments, the dose is subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In one embodiment, the present disclosure provides a method of treating a receptor-interacting protein kinase 1-mediated disease or disorder, comprising administering to a subject in need thereof at a dose of about 5 to 60 mg per day of Compound 1, or a pharmaceutically acceptable salt thereof.

In one embodiment, the present disclosure provides a method of treating amyotrophic lateral sclerosis, comprising administering to a subject in need thereof at a dose of about 5 to 60 mg per day of Compound 1, or a pharmaceutically acceptable salt thereof.

In one embodiment, the present disclosure provides a method of treating multiple sclerosis, comprising administering to a subject in need thereof at a dose of about 5 to 60 mg per day of Compound 1, or a pharmaceutically acceptable salt thereof.

In one embodiment, the dose is about 10 mg per day of Compound 1, or a pharmaceutically acceptable salt thereof.

In one embodiment, the dose is about 20 mg per day of Compound 1, or a pharmaceutically acceptable salt thereof.

In one embodiment, the dose is about 30 mg per day of Compound 1, or a pharmaceutically acceptable salt thereof.

In one embodiment, the dose is about 40 mg per day of Compound 1, or a pharmaceutically acceptable salt thereof.

In one embodiment, the dose is about 10 mg twice a day of Compound 1, or a pharmaceutically acceptable salt thereof.

In one embodiment, the dose is about 20 mg twice a day of Compound 1, or a pharmaceutically acceptable salt thereof.

In one embodiment, the dose is about 15 mg twice a day of Compound 1, or a pharmaceutically acceptable salt thereof.

In one embodiment, the present disclosure provides a method of treating amyotrophic lateral sclerosis, comprising administering to a subject in need thereof at a dose of about 20 mg of Compound 1, or a pharmaceutically acceptable salt thereof.

In one embodiment, the present disclosure provides a method of treating amyotrophic lateral sclerosis, comprising administering to a subject in need thereof at a dose of about 20 mg of Compound 1.

In one embodiment, the present disclosure provides a method of treating amyotrophic lateral sclerosis, comprising administering to a subject in need thereof at a dose of about 20 mg per day of Compound 1, or a pharmaceutically acceptable salt thereof.

In one embodiment, the present disclosure provides a method of treating amyotrophic lateral sclerosis, comprising administering to a subject in need thereof at a dose of about 20 mg per day of Compound 1.

In one embodiment, the present disclosure provides a method of treating amyotrophic lateral sclerosis, comprising administering to a subject in need thereof at a dose of about 20 mg twice a day of Compound 1, or a pharmaceutically acceptable salt thereof.

In one embodiment, the present disclosure provides a method of treating amyotrophic lateral sclerosis, comprising administering to a subject in need thereof at a dose of about 20 mg twice a day of Compound 1.

In one embodiment, the present disclosure provides a method of treating amyotrophic lateral sclerosis, comprising administering to a subject in need thereof at a dose of about 15 mg of Compound 1, or a pharmaceutically acceptable salt thereof.

In one embodiment, the present disclosure provides a method of treating amyotrophic lateral sclerosis, comprising administering to a subject in need thereof at a dose of about 15 mg twice a day of Compound 1, or a pharmaceutically acceptable salt thereof.

In one embodiment, the present disclosure provides a method of treating amyotrophic lateral sclerosis, comprising administering to a subject in need thereof at a dose of about 15 mg twice a day of Compound 1.

In one embodiment, the present disclosure provides a method of treating multiple sclerosis, comprising administering to a subject in need thereof at a dose of about 20 mg per day of Compound 1, or a pharmaceutically acceptable salt thereof.

In one embodiment, the present disclosure provides a method of treating multiple sclerosis, comprising administering to a subject in need thereof at a dose of about 20 mg per day of Compound 1.

In one embodiment, the present disclosure provides a method of multiple sclerosis, comprising administering to a subject in need thereof at a dose of about 20 mg twice a day of Compound 1, or a pharmaceutically acceptable salt thereof.

In one embodiment, the present disclosure provides a method of treating multiple sclerosis, comprising administering to a subject in need thereof at a dose of about 15 mg of Compound 1, or a pharmaceutically acceptable salt thereof.

In one embodiment, the present disclosure provides a method of treating multiple sclerosis, comprising administering to a subject in need thereof at a dose of about 15 mg twice a day of Compound 1, or a pharmaceutically acceptable salt thereof.

In one embodiment, the present disclosure provides a method of treating a receptor-interacting protein kinase 1-mediated disease or disorder comprising administering to a subject in need thereof at a dose of about 10-40 mg per day of Compound 1, or a pharmaceutically acceptable salt thereof wherein said compound is administered in an amount to provide maximum median peripheral pS166-RIPK1 inhibition in peripheral blood mononuclear cells (PBMCs) lysates ranged between 82%-97% assessed by Meso Scale Discovery (MSD) assay.

In one embodiment, the present disclosure provides a method of treating amyotrophic lateral sclerosis comprising administering to a subject in need thereof at a dose of about 10-40 mg per day of Compound 1, or a pharmaceutically acceptable salt thereof; wherein said compound is administered in an amount to provide maximum median peripheral pS166-RIPK1 inhibition in peripheral blood mononuclear cells (PBMCs) lysates ranged between 82%-97% assessed by Meso Scale Discovery (MSD) assay.

In one embodiment, the present disclosure provides a method of treating multiple sclerosis comprising administering to a subject in need thereof at a dose of about 10-40 mg per day of Compound 1, or a pharmaceutically acceptable salt thereof; wherein said compound is administered in an amount to provide maximum median peripheral pS166-RIPK1 inhibition in peripheral blood mononuclear cells (PBMCs) lysates ranged between 82%-97% assessed by Meso Scale Discovery (MSD) assay.

In one embodiment, the present disclosure provides a method of treating a receptor-interacting protein kinase 1-mediated disease or disorder comprising administering to a subject in need thereof at a dose of about 10-40 mg per day of Compound 1, or a pharmaceutically acceptable salt thereof; wherein said compound is administered to provide absorption with median tₘₐₓ reached between 1-2 hours post-administering to the subject in a fasted state.

In one embodiment, the present disclosure provides a method of treating a receptor-interacting protein kinase 1-mediated disease or disorder comprising administering to a subject in need thereof at a dose of about 10-40 mg per day of Compound 1, or a pharmaceutically acceptable salt thereof; wherein said compound is administered to provide absorption with median tₘₐₓ reached between about 0.5-5 hours post-administering to the subject in a fed state.

In one embodiment, the present disclosure provides a method of treating a receptor-interacting protein kinase 1-mediated disease or disorder comprising administering to a subject in need thereof at a dose of about 10-40 mg per day of Compound 1, or a pharmaceutically acceptable salt thereof; wherein said compound is administered in an amount to provide a mean ratio CSF to unbound plasma concentrations was about 0.8 and 1.3 in the time intervals of post administering to the subject in a fed state.

In one embodiment, the present disclosure provides a method of treating a receptor-interacting protein kinase 1-mediated disease or disorder comprising administering to a subject in need thereof at a dose of about 10-40 mg per day of Compound 1, or a pharmaceutically acceptable salt thereof; wherein said compound is administered to provide a median steady state within 1.5-2 days after administration.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a oral plasma clearance of about 8.53 L/h.

In one embodiment, the present disclosure provides a method of treating a receptor-interacting protein kinase 1-mediated disease or disorder comprising administering to a subject in need thereof a therapeutically effective amount of Compound 1, or a pharmaceutically acceptable salt thereof; wherein said compound is administered in an amount to provide a mean AUCₜₐᵤ of about 1370 to about 2220 h*ng/mL; and a mean Cₘₐₓ of about 209 to about 359 ng/mL.

In one embodiment, the present disclosure provides a method of treating a receptor-interacting protein kinase 1-mediated disease or disorder comprising administering about 20 mg twice a day of Compound 1 to a subject in need thereof, wherein said compound is administered to provide a mean AUCₜₐᵤ of about 1370 to about 2220 h*ng/mL; and a mean Cₘₐₓ of about 209 to about 359 ng/mL.

In one embodiment, the present disclosure provides a method of treating a receptor-interacting protein kinase 1-mediated disease or disorder comprising administering about 20 mg twice a day of Compound 1 or an equivalent amount of a pharmaceutically acceptable salt thereof to a subject in need thereof, wherein said compound is administered to provide a mean AUCₜₐᵤ of about 1370 to about 2220 h*ng/mL; and a mean Cₘₐₓ of about 209 to about 359 ng/mL.

In one embodiment, the present disclosure provides a method of treating a receptor-interacting protein kinase 1-mediated disease or disorder comprising administering a therapeutically effective amount of Compound 1 to a subject in need thereof, wherein said compound is administered in an amount to provide a mean AUCₜₐᵤ of about 1370 to about 2220 h*ng/mL.

In one embodiment, the present disclosure provides a method of treating a receptor-interacting protein kinase 1-mediated disease or disorder comprising administering a therapeutically effective amount of Compound 1 to a subject in need thereof, wherein said compound is administered in an amount to provide a mean Cₘₐₓ of about 209 to about 359 ng/mL.

In one embodiment, the present disclosure provides a method of treating amyotrophic lateral sclerosis comprising administering to a subject in need thereof a therapeutically effective amount of Compound 1, or a pharmaceutically acceptable salt thereof; wherein said compound is administered in an amount to provide a mean AUCₜₐᵤ of about 1370 to about 2220 h*ng/mL; and a mean Cₘₐₓ of about 209 to about 359 ng/mL.

In one embodiment, the present disclosure provides a method of treating amyotrophic lateral sclerosis comprising administering about 20 mg twice a day of Compound 1 or an equivalent amount of a pharmaceutically acceptable salt thereof to a subject in need thereof, wherein said compound is administered to provide a mean AUCₜₐᵤ of about 1370 to about 2220 h*ng/mL; and a mean Cₘₐₓ of about 209 to about 359 ng/mL.

In one embodiment, the present disclosure provides a method of treating amyotrophic lateral sclerosis comprising administering a therapeutically effective amount of Compound 1 to a subject in need thereof, wherein said compound is administered in an amount to provide a mean AUCₜₐᵤ of about 1370 to about 2220 h*ng/mL.

In one embodiment, the present disclosure provides a method of treating amyotrophic lateral sclerosis comprising administering a therapeutically effective amount of Compound 1 to a subject in need thereof, wherein said compound is administered in an amount to provide a mean Cₘₐₓ of about 209 to about 359 ng/mL.

In one embodiment, the present disclosure provides a method of treating multiple sclerosis comprising administering to a subject in need thereof a therapeutically effective amount of Compound 1, or a pharmaceutically acceptable salt thereof; wherein said compound is administered in an amount to provide a mean AUCₜₐᵤ of about 1370 to about 2220 h*ng/mL; and a mean Cₘₐₓ of about 209 to about 359 ng/mL.

In one embodiment, the present disclosure provides a method of treating multiple sclerosis comprising administering about 20 mg twice a day of Compound 1 or an equivalent amount of a pharmaceutically acceptable salt thereof to a subject in need thereof, wherein said compound is administered to provide a mean AUCₜₐᵤ of about 1370 to about 2220 h*ng/mL; and a mean Cₘₐₓ of about 209 to about 359 ng/mL.

In one embodiment, the present disclosure provides a method of treating multiple sclerosis comprising administering a therapeutically effective amount of Compound 1 to a subject in need thereof, wherein said compound is administered in an amount to provide a mean AUCₜₐᵤ of about 1370 to about 2220 h*ng/mL.

In one embodiment, the present disclosure provides a method of treating multiple sclerosis comprising administering a therapeutically effective amount of Compound 1 to a subject in need thereof, wherein said compound is administered in an amount to provide a mean Cₘₐₓ of about 209 to about 359 ng/mL.

In one embodiment, the present disclosure provides a method of treating a receptor-interacting protein kinase 1-mediated disease or disorder comprising administering to a subject in need thereof a therapeutically effective amount of compound 1 or a pharmaceutically acceptable salt thereof; wherein said compound is administered in an amount to provide a mean AUCₜₐᵤ of about 1910 to about 2170 h*ng/mL; and a mean Cₘₐₓ of about 245 to about 255 ng/mL.

In one embodiment, the present disclosure provides a method of treating amyotrophic lateral sclerosis comprising administering to a subject in need thereof a therapeutically effective amount of compound 1 or a pharmaceutically acceptable salt thereof; wherein said compound is administered in an amount to provide a mean AUCₜₐᵤ of about 1910 to about 2170 h*ng/mL; and a mean Cₘₐₓ of about 245 to about 255 ng/mL.

In one embodiment, the present disclosure provides a method of treating multiple sclerosis comprising administering to a subject in need thereof a therapeutically effective amount of compound 1 or a pharmaceutically acceptable salt thereof; wherein said compound is administered in an amount to provide a mean AUCₜₐᵤ of about 1910 to about 2170 h*ng/mL; and a mean Cₘₐₓ of about 245 to about 255 ng/mL.

In one embodiment, the present disclosure provides a method of treating a receptor-interacting protein kinase 1-mediated disease or disorder comprising administering to a subject in need thereof about 20 mg once a day of Compound 1 or a pharmaceutically acceptable salt thereof; wherein said compound is administered to provide a mean AUCₜₐᵤ of about 1910 to about 2170 h*ng/mL; and a mean Cₘₐₓ of about 245 to about 255 ng/mL.

In one embodiment, the present disclosure provides a method of treating amyotrophic lateral sclerosis comprising administering to a subject in need thereof about 20 mg once a day of Compound 1 or a pharmaceutically acceptable salt thereof; wherein said compound is administered to provide a mean AUCₜₐᵤ of about 1910 to about 2170 h*ng/mL; and a mean Cₘₐₓ of about 245 to about 255 ng/mL.

In one embodiment, the present disclosure provides a method of treating multiple sclerosis comprising administering to a subject in need thereof about 20 mg once a day of Compound 1 or a pharmaceutically acceptable salt thereof; wherein said compound is administered to provide a mean AUCₜₐᵤ of about 1910 to about 2170 h*ng/mL; and a mean Cₘₐₓ of about 245 to about 255 ng/mL.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a mean AUCₜₐᵤ of about 1910 to about 2170 h*ng/mL.

In one embodiment, the present disclosure provides a method of treating amyotrophic lateral sclerosis comprising administering to a subject in need thereof a therapeutically effective amount of Compound 1 or a pharmaceutically acceptable salt thereof; wherein said compound is administered in an amount to provide a mean AUCₜₐᵤ of about 1910 to about 2170 h*ng/mL.

In one embodiment, the present disclosure provides a method of treating multiple sclerosis comprising administering to a subject in need thereof a therapeutically effective amount of Compound 1 or a pharmaceutically acceptable salt thereof; wherein said compound is administered in an amount to provide a mean AUCₜₐᵤ of about 1910 to about 2170 h*ng/mL.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a a mean Cₘₐₓ of about 245 to about 255 ng/mL.

In one embodiment, the present disclosure provides a method of treating amyotrophic lateral sclerosis comprising administering to a subject in need thereof a therapeutically effective amount of Compound 1 or a pharmaceutically acceptable salt thereof; wherein said compound is administered in an amount to provide a a mean Cₘₐₓ of about 245 to about 255 ng/mL.

In one embodiment, the present disclosure provides a method of multiple sclerosis comprising administering to a subject in need thereof a therapeutically effective amount of Compound 1 or a pharmaceutically acceptable salt thereof; wherein said compound is administered in an amount to provide a a mean Cₘₐₓ of about 245 to about 255 ng/mL.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a mean AUCₜₐᵤ of about 1150 to about 1710 h*ng/mL; and a mean Cₘₐₓ of about 229 to about 284 ng/mL.

In one embodiment, the present disclosure provides a method of treating a receptor-interacting protein kinase 1-mediated disease or disorder comprising administering to a subject in need thereof about 15 mg twice a day of Compound 1 or a pharmaceutically acceptable salt thereof; wherein said compound is administered to provide a mean AUCₜₐᵤ of about 1150 to about 1710 h*ng/mL; and a mean Cₘₐₓ of about 229 to about 284 ng/mL.

In one embodiment, the present disclosure provides a method of treating a receptor-interacting protein kinase 1-mediated disease or disorder comprising administering to a subject in need thereof about 15 mg twice a day of Compound 1 or a pharmaceutically acceptable salt thereof; wherein said compound is administered in an amount to provide a mean AUCₜₐᵤ of about 1150 to about 1710 h*ng/mL.

In one embodiment, the present disclosure provides a method of treating a receptor-interacting protein kinase 1-mediated disease or disorder comprising administering to a subject in need thereof about 15 mg twice a day of Compound 1 or a pharmaceutically acceptable salt thereof; wherein said compound is administered in an amount to provide a a mean Cₘₐₓ of about 229 to about 284 ng/mL.

In one embodiment, the present disclosure provides a method of treating amyotrophic lateral sclerosis comprising administering to a subject in need thereof a therapeutically effective amount of Compound 1 or a pharmaceutically acceptable salt thereof; wherein said compound is administered in an amount to provide a mean AUCₜₐᵤ of about 1150 to about 1710 h*ng/mL; and a mean Cₘₐₓ of about 229 to about 284 ng/mL.

In one embodiment, the present disclosure provides a method of treating amyotrophic lateral sclerosis comprising administering to a subject in need thereof about 15 mg twice a day of Compound 1 or a pharmaceutically acceptable salt thereof; wherein said compound is administered to provide a mean AUCₜₐᵤ of about 1150 to about 1710 h*ng/mL; and a mean Cₘₐₓ of about 229 to about 284 ng/mL.

In one embodiment, the present disclosure provides a method of treating amyotrophic lateral sclerosis comprising administering to a subject in need thereof about 15 mg twice a day of Compound 1 or a pharmaceutically acceptable salt thereof; wherein said compound is administered in an amount to provide a mean AUCₜₐᵤ of about 1150 to about 1710 h*ng/mL.

In one embodiment, the present disclosure provides a method of treating amyotrophic lateral sclerosis comprising administering to a subject in need thereof about 15 mg twice a day of Compound 1 or a pharmaceutically acceptable salt thereof; wherein said compound is administered in an amount to provide a a mean Cₘₐₓ of about 229 to about 284 ng/mL.

In one embodiment, the present disclosure provides a method of treating multiple sclerosis comprising administering to a subject in need thereof a therapeutically effective amount of Compound 1 or a pharmaceutically acceptable salt thereof; wherein said compound is administered in an amount to provide a mean AUCₜₐᵤ of about 1150 to about 1710 h*ng/mL; and a mean Cₘₐₓ of about 229 to about 284 ng/mL.

In one embodiment, the present disclosure provides a method of multiple sclerosis comprising administering to a subject in need thereof about 15 mg twice a day of Compound 1 or a pharmaceutically acceptable salt thereof; wherein said compound is administered to provide a mean AUCₜₐᵤ of about 1150 to about 1710 h*ng/mL; and a mean Cₘₐₓ of about 229 to about 284 ng/mL.

In one embodiment, the present disclosure provides a method of treating multiple sclerosis comprising administering to a subject in need thereof about 15 mg twice a day of Compound 1 or a pharmaceutically acceptable salt thereof; wherein said compound is administered in an amount to provide a mean AUCₜₐᵤ of about 1150 to about 1710 h*ng/mL.

In one embodiment, the present disclosure provides a method of treating multiple sclerosis comprising administering to a subject in need thereof about 15 mg twice a day of Compound 1 or a pharmaceutically acceptable salt thereof; wherein said compound is administered in an amount to provide a a mean Cₘₐₓ of about 229 to about 284 ng/mL.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a mean AUC₀₋₂₄ no more than about 15 μM*h.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a mean Cₘₐₓ no more than about 1.3 μM.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a mean Cₘₐₓ no more than about 3.0 μM and a mean AUC₀₋₂₄ no more than about 20 μM*h.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein said compound is administered in an amount to provide a mean Cₘₐₓ no more than about 1.0 μM and a mean AUC₀₋₂₄ no more than about 9.0 μM*h.

In one embodiment, the present disclosure provides a method of treating a receptor-interacting protein kinase 1-mediated disease or disorder comprising administering to a subject in need thereof a therapeutically effective amount of compound 1 or a pharmaceutically acceptable salt thereof; wherein said compound is administered in an amount to provide a mean Cₘₐₓ no more than about 1.3 μM and a mean AUC₀₋₂₄ no more than about 15 μM*h.

In one embodiment, the present disclosure provides Compound 1, or a pharmaceutically acceptable salt thereof is used in the treatment of a receptor-interacting protein kinase 1-mediated disease or disorder at a dose of about 5 to 60 mg per day.

In one embodiment, the present disclosure provides Compound 1, or a pharmaceutically acceptable salt thereof is used in the treatment of a neurodegenerative disease at a dose of about 5 to 60 mg per day.

In one embodiment, the present disclosure provides Compound 1, or a pharmaceutically acceptable salt thereof is used in the treatment of amyotrophic lateral sclerosis at a dose of about 5 to 60 mg per day.

In one embodiment, the present disclosure provides Compound 1, or a pharmaceutically acceptable salt thereof is used in the treatment of multiple disease at a dose of about 5 to 60 mg per day.

In one embodiment, the present disclosure provides Compound 1, or a pharmaceutically acceptable salt thereof is used in the treatment of amyotrophic lateral sclerosis at a dose of about 20 mg twice a day.

In one embodiment, the present disclosure provides Compound 1 is used in the treatment of amyotrophic lateral sclerosis at a dose of about 20 mg twice a day.

In one embodiment, the present disclosure provides Compound 1 is used in the treatment of amyotrophic lateral sclerosis at a dose of about 15 mg twice a day.

In one embodiment, the present disclosure provides Compound 1, or a pharmaceutically acceptable salt thereof is used in the treatment of amyotrophic lateral sclerosis at a dose of about 15 mg twice a day.

In one embodiment, the present disclosure provides Compound 1, or a pharmaceutically acceptable salt thereof is used in the treatment of multiple sclerosis at a dose of about 15 mg twice a day.

In one embodiment, the present disclosure provides Compound 1 is used in the treatment of multiple sclerosis at a dose of about 15 mg twice a day.

In one embodiment, the present disclosure provides Compound 1, or a pharmaceutically acceptable salt thereof is used in the treatment of amyotrophic lateral sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides Compound 1 is used in the treatment of amyotrophic lateral sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides Compound 1, or a pharmaceutically acceptable salt thereof is used in the treatment of multiple sclerosis at a dose of about 20 mg per day.

In one embodiment, the present disclosure provides Compound 1 is used in the treatment of multiple sclerosis at a dose of about 20 mg per day.

In one embodiment, the present disclosure provides Compound 1, or a pharmaceutically acceptable salt thereof is used in the treatment of multiple sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides Compound 1 is used in the treatment of multiple sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides Compound 1, or a pharmaceutically acceptable salt thereof is used in the treatment of amyotrophic lateral sclerosis at a dose of about 20 mg per day.

In one embodiment, the present disclosure provides Compound 1 is used in the treatment of amyotrophic lateral sclerosis at a dose of about 20 mg per day.

In one embodiment, the present disclosure provides Compound 1, or a pharmaceutically acceptable salt thereof is used in the treatment of amyotrophic lateral sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides Compound 1 is used in the treatment of amyotrophic lateral sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides Compound 1, or a pharmaceutically acceptable salt thereof is used in the treatment of multiple sclerosis at a dose of about 20 mg twice a day.

In one embodiment, the present disclosure provides Compound 1 is used in the treatment of multiple sclerosis at a dose of about 20 mg twice a day.

In one embodiment, the present disclosure provides Compound 1, or a pharmaceutically acceptable salt thereof is used in the treatment of multiple sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides Compound 1 is used in the treatment of multiple sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides use of Compound 1, or a pharmaceutically acceptable salt thereof for preparing a medicament for the treatment of a receptor-interacting protein kinase 1-mediated disease or disorder at a dose of about 5 to 60 mg per day.

In one embodiment, the present disclosure provides use of Compound 1, or a pharmaceutically acceptable salt thereof for preparing a medicament for the treatment of a neurodegenerative disease at a dose of about 5 to 60 mg per day.

In one embodiment, the present disclosure provides use of Compound 1, or a pharmaceutically acceptable salt thereof for preparing a medicament for the treatment of amyotrophic lateral sclerosis at a dose of about 5 to 60 mg per day.

In one embodiment, the present disclosure provides use of Compound 1, or a pharmaceutically acceptable salt thereof for preparing a medicament for the treatment of multiple disease at a dose of about 5 to 60 mg per day.

In one embodiment, the present disclosure provides use of Compound 1, or a pharmaceutically acceptable salt thereof for preparing a medicament for the treatment of amyotrophic lateral sclerosis at a dose of about 20 mg twice a day.

In one embodiment, the present disclosure provides use of Compound 1 for preparing a medicament for the treatment of amyotrophic lateral sclerosis at a dose of about 20 mg twice a day.

In one embodiment, the present disclosure provides use of Compound 1, or a pharmaceutically acceptable salt thereof for preparing a medicament for the treatment of amyotrophic lateral sclerosis at a dose of about 15 mg twice a day.

In one embodiment, the present disclosure provides use of Compound 1, or a pharmaceutically acceptable salt thereof for preparing a medicament for the treatment at a dose of about 15 mg twice a day.

In one embodiment, the present disclosure provides use of Compound 1, or a pharmaceutically acceptable salt thereof for preparing a medicament for the treatment of amyotrophic lateral sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides use of Compound 1 for preparing a medicament for the treatment of amyotrophic lateral sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides use of Compound 1, or a pharmaceutically acceptable salt thereof for preparing a medicament for the treatment of multiple sclerosis at a dose of about 20 mg per day.

In one embodiment, the present disclosure provides use of Compound 1 for preparing a medicament for the treatment of multiple sclerosis at a dose of about 20 mg per day.

In one embodiment, the present disclosure provides use of Compound 1, or a pharmaceutically acceptable salt thereof for preparing a medicament for the treatment of multiple sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides use of Compound 1 for preparing a medicament for the treatment of multiple sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides use of Compound 1, or a pharmaceutically acceptable salt thereof for preparing a medicament for the treatment of amyotrophic lateral sclerosis at a dose of about 20 mg per day.

In one embodiment, the present disclosure provides use of Compound 1, or a pharmaceutically acceptable salt thereof for preparing a medicament for the treatment of amyotrophic lateral sclerosis at a dose of about 20 mg per day to provide a mean AUCₜₐᵤ of about 1910 to about 2170 h*ng/mL; and a mean Cₘₐₓ of about 245 to about 255 ng/mL.

In one embodiment, the present disclosure provides use of Compound 1, or a pharmaceutically acceptable salt thereof for preparing a medicament for the treatment of multiple sclerosis at a dose of about 20 mg twice a day.

In one embodiment, the present disclosure provides use of Compound 1, or a pharmaceutically acceptable salt thereof for preparing a medicament for the treatment of multiple sclerosis at a dose of about 20 mg twice a day to provide a mean AUCₜₐᵤ of about 1370 to about 2220 h*ng/mL; and a mean Cₘₐₓ of about 209 to about 359 ng/mL.

In one embodiment, the present disclosure provides an article of manufacture, a packaging, or an administration unit, comprising:
a packaging material;
the above defined pharmaceutical composition; and
a label or package insert contained within said packaging material, indicating that said pharmaceutical composition is administered to a patient for the treatment of a receptor-interacting protein kinase 1-mediated disease or disorder, at a dose of about 5 to 60 mg per day of 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof.

In one embodiment, the present disclosure provides a dose of about 5 to 60 mg per day of 4-(3,3-difluoro-2,2-dimethyl-propanoyl)-3,5-dihydro-2H-pyrido[3,4-f] [1,4]oxazepine-9-carbonitrile, or a pharmaceutically acceptable salt thereof, for the manufacture of a medicament useful in treating a receptor-interacting protein kinase 1-mediated disease or disorder.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein the effective amount of the compound is about 10-40 mg of Compound 1, wherein the dose is administered with food.

In one embodiment, the present disclosure provides a method of treating a receptor-interacting protein kinase 1-mediated disease or disorder comprising administering to a subject in need thereof a therapeutically effective amount of Compound 1 or a pharmaceutically acceptable salt thereof; wherein the effective amount of the compound is about 10-40 mg of Compound 1, wherein the dose is administered without food.

In some embodiments, the subject is a mammal.

In some embodiments, the mammal is a human.

In some embodiments, the dose is administered orally.

In some embodiments, the dose is administered in a form of tablets.

In some embodiments, the dose is administered in the form of pills, capsules, semisolids, powders, sustained release formulations, solutions, suspensions, elixirs, aerosols, or any other appropriate compositions.

In some embodiments, the subject is administered the RIPK1 inhibitor compound for a period of about 4, 8, 12, 16, 20 or 24 weeks. In some embodiments, the subject is administered the RIPK1 inhibitor compound for a period of about 12 weeks.

In some embodiments, the dose is administered with food. In some embodiments, the dose is administered once daily with food. In some embodiments, the dose of 5 mg, 15 mg, 30 mg, 40 mg or 60 mg is administered with food. In some embodiments, the dose of 5 mg, 10 mg, 15 mg, 20 mg, 30 mg, 40 mg, or 60 mg is administered once daily with food. In some embodiments, the dose of 20 mg is administered once daily with food. In some embodiments, the dose of 20 mg is administered twice a day with food.

In some embodiments, the dose is administered in oral solution or tablets. In some embodiments, the dose is administered in oral solution or tablets with food. In some embodiments, the dose is administered once daily in oral solution or tablets. In some embodiments, the dose is administered once daily in oral solution or tablets with food. In some embodiments, the dose of 20 mg is administered once daily in oral solution or tablets. In some embodiments, the dose of 20 mg is administered twice a day in oral solution or tablets with food. In some embodiments, the dose of 15 mg is administered twice a day in oral solution or tablets.

In some embodiments, the dose is administered without food. In some embodiments, the dose is administered once daily without food. In some embodiments, the dose of 5 mg, 15 mg, 30 mg, 40 mg or 60 mg is administered without food. In some embodiments, the dose of 5 mg, 10 mg, 15 mg, 20 mg, 30 mg, 40 mg, or 60 mg is administered once daily without food. In some embodiments, the dose of 20 mg is administered once daily without food. In some embodiments, the dose of 20 mg is administered twice a day without food.

In some embodiments, the dose is administered in oral solution or tablets. In some embodiments, the dose is administered in oral solution or tablets without food. In some embodiments, the dose is administered once daily in oral solution or tablets without food. In some embodiments, the dose is administered once daily in oral solution or tablets without food. In some embodiments, the dose of 20 mg is administered once daily in oral solution or tablets. In some embodiments, the dose of 20 mg is administered twice a day in oral solution or tablets without food. In some embodiments, the dose of 15 mg is administered twice a day in oral solution or tablets without food.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

### Abbreviations

AE: adverse event
AESI: adverse event of special interest
ALS: amyotrophic lateral sclerosis
ALSAQ-5: Amyotrophic Lateral Sclerosis Assessment Scales-5 items
ALSFRS-R: Amyotrophic Lateral Functional Rating Scale Revised
ALT: alanine aminotransferase
AST: aspartate aminotransferase
AUC: area under the curve
AUCₗₐₛₜ: area under the plasma concentration versus time curve from time zero to the real time tₗₐₛₜ
BID: Bis In Die; twice daily
CAFS: combined assessment of function and survival
CI: confidence interval
CL/F: apparent total body clearance of a drug from the plasma
Cₘₐₓ: maximum plasma concentration
CNS: central nervous system
CPK: creatine phosphokinase
CSF: cerebrospinal fluid
C-SSRS: Columbia Suicide Severity Rating Scale
CYP: cytochrome P450
ECG: electrocardiogram
FDA: Food and Drug Administration
GM: Group mean
HIV: human immunodeficiency virus
IC₅₀ half maximal inhibitory concentration
ICH: International Council for Harmonisation
IMP: investigational medicinal product
IV: Intravenous
LC-MS/MS: liquid chromatography tandem mass spectrometry
MAD: multiple ascending dose
MLKL: mixed lineage kinase domain-like pseudokinase
MS: multiple sclerosis
ML: neurofilament light chain
NOAEL: no-observed-adverse-effect-level
OD: once daily
PBMC: peripheral blood mononuclear cell
PBPK: physiological-based pharmacokinetic modeling and simulation
PCSA: potentially clinically significant abnormality
PD: pharmacodynamic
PK: pharmacokinetic
PRO: patient-reported outcome
PT: preferred term
PT: Prothrombin Time
QD: Once daily
RIPK1: receptor-interacting serine/threonine-protein kinase 1
RIPK3: receptor-interacting serine/threonine-protein kinase 3
SAD: single ascending dose
SAE: serious adverse event
SD: standard deviation
SE: standard error
t_{1/2} half life
t_{1/2z} terminal half-life
tₘₐₓ time taken to reach the maximum concentration
TEAE: treatment-emergent adverse event
TNF-α: tumor necrosis factor alpha
tₗₐₛₜ: time corresponding to the last concentration above the limit of quantification
ULN: upper limit of normal
Vz/F: apparent volume of distribution during the terminal phase

### EXAMPLES

The following examples are provided to illustrate certain disclosed embodiments and are not to be construed as limiting the scope of this disclosure in any way. In the Examples discussed below, the RIPK1 inhibitor, as defined above, may be also referred as “Compound 1”, “the compound” or “the drug” interchangeably.

### Example 1: A Randomized, Double-Blind, Placebo-Controlled Study of the Safety, Tolerability and Pharmacokinetics of Ascending Single and Repeated Oral Doses of Compound 1 in Healthy Adult Participants

This single ascending dose (SAD) study consisted of three parts: Part 1a, which is a phase 1, randomized, placebo-controlled, double-blind, sequential ascending single dose study; Part 1b, which is phase 1, open-label, sequential single dose study; and Part 2, which is an ascending 14-day repeated oral dose study.

For Part 1a, the primary objective is to assess the safety and tolerability of Compound 1 after single ascending oral doses in healthy adult participants. The secondary objective is to determine the pharmacokinetic (PK) parameters of Compound 1 in plasma samples after ascending single oral doses of Compound 1 in healthy adult participants. The exploratory objective is to evaluate the pharmacodynamic (PD) effect of Compound 1 on the inhibition of phosphorylated serine 166 (pS166)-RIPK1 in peripheral blood mononuclear cell (PBMC) lysates from healthy adult participants, and to determine the PK parameters of Compound 1 in urine samples after ascending single oral doses of Compound 1.

For Part 1b, the secondary objectives are to determine the PK parameters of Compound 1 in plasma and to measure Compound 1 concentrations in CSF samples after single oral dose, and to assess safety and tolerability of Compound 1 after single oral dose.

For Part 2, the primary objective is to assess the tolerability and safety of 14-day ascending repeated oral doses of Compound 1 in healthy adult participants. The secondary objectives are to determine the PK parameters of Compound 1 in plasma sample after ascending repeated oral doses; and to assess the potential for CYP3A4 enzyme induction by Compound 1 after repeated doses. The exploratory objectives are to evaluate the PD effect of Compound 1 on the inhibition of pS166 RIPK1 in PBMC lysates and to determine the PK parameters of Compound 1 in urine samples after ascending repeated oral doses of Compound 1, if needed, based on Part 1a and Part 1b results.

Part 1a evaluated 4 sequential ascending dose levels of Compound 1 under fasted conditions: 10 mg, 20 mg, 30 mg, and 40 mg, each dose per cohort. Participants were fasted overnight for at least 8 hours before the morning dose and 2 hours after dosing.

Part 1b is an open-label study with two single dose levels of Compound 1, previously tested for safety/tolerability in SAD cohorts of Part 1a, were selected (10 mg and 40 mg) and sequentially administered under a fed condition, each dose per cohort. Participants were fasted overnight for at least 8 hours, and then fed with a light breakfast approximately 30 minutes before Compound 1 dosing. In both Part 1a and 1b, all participants received one single dose of treatment.

Part 2 was a randomized, double-blind, placebo-controlled study that evaluated 14-day multiple ascending oral doses of Compound 1 once daily (OD) or twice daily (BID) regimen under fasted conditions: 10 mg QD, 20 mg QD, 15 mg BID, and 20 mg BID. Participants fasted overnight for at least 8 hours before the morning dose and 2 hours after dosing on Day 1 and Day 14. All other morning doses were administered after an overnight fast and approximately 30 minutes before a breakfast snack. Sentinel dosing approach was followed where two of ten participants in each cohort were dosed the first day (n=1, Compound; n=1, placebo) in advance of the full cohort. The remaining 8 participants from each cohort were randomized to receive either Compound or placebo (randomized 7:1) on the following days with at least 48-hour interval from dosing of the sentinel participants, allowing a safety evaluation at the predicted steady state.

The study duration included a 28-day screening period, a treatment period (1 day each for Parts 1a and 1b, 14 days for Part 2), followed by an observation period (1-2 days for SAD and PK CSF Part, 3 days for MAD-Part 2), and a follow up with the end of study (EOS) visit between 4-6 days after dosing in Parts 1a and 1b and 5-7 days after the last administration in Part 2.

Eligible participants in both studies were healthy men or women, 18-55 years of age, with body weight between 50-100 kg for men or 40-90 kg for women, and a body mass index between 18-30 kg/m2.

Safety and Tolerability Assessment

Safety and tolerability were assessed by physical examination, neurological examination, monitoring adverse events (AEs) and potentially clinically significant abnormalities (PCSAs) in clinical laboratory tests (hematology, clinical chemistry, and urinalysis), vital signs (body temperature, blood pressure and heart rate), and ECG parameters (heart rate, RR, PR, QRS, QT, QTcF, QTcB intervals) measured in the standard 12-lead ECG or ECGs extracted from 24-hour Holter ECG data.

Plasma and CSF PK Parameters

The following plasma PK parameters were calculated from plasma Compound 1 concentrations obtained after single (Part 1a) or repeated (Part 2) oral doses: maximum plasma concentration observed (Cₘₐₓ); time to reach Cₘₐₓ (tₘₐₓ); interval between administration time and the sampling time preceding the first concentration above the limit of quantification (t_{lag}); area under the plasma concentration versus time curve from time zero to the real time (AUCₗₐₛₜ); area under the plasma concentration versus time curve extrapolated to infinity (AUC); area under the concentration versus time curve during a dosage interval (tau), with tau=12 hours for BID regimen and tau=24 hours for QD regimen terminal (AUCₜₐᵤ); half-life associated with the terminal slope (t_{1/2z}); apparent total body clearance of a drug from the plasma CL/F; apparent total body clearance after repeated extra vascular doses of a drug at steady state (CLss/F); the apparent volume of distribution (Vz/F); concentration observed just before treatment administration during repeated dosing (C_{trough}).

In Part 1a, plasma samples were collected before dosing and at 10 post-dose timepoints on Day 1 (30 minutes, 1 hour, followed by every hour until 6 hours, and then every 2 hours until 12 hours), and single samples on Day 2 (at 24 hours) and Day 3 (at 48 hours). In Part 1b, timepoints included at least pre-dose and at 10 post-dose timepoints, including plasma sampling at the same time of CSF sampling. In Part 2, on Day 1 and Day 14, plasma samples were collected at pre-dose and at 10 to 11 post-dose timepoints. In addition, plasma samples were collected on Days 2, 3, 5, 7, 11, 15, 16, and 17.

Compound 1 concentrations in CSF were measured after single oral dosing in two dedicated cohorts in Part 1b. Lumbar puncture was performed for CSF collection and only one CSF sample per participant was collected on Day 1. Given the limited number of CSF PK samples that were collected in this study, CSF PK parameters were not determined; instead, Compound 1 concentrations in CSF were reported and concentration ratio between CSF and unbound plasma was calculated.

The plasma 4β-hydroxycholesterol on Day 14 versus Day 1 pre-dose concentration ratios were calculated to evaluate the potential for CYP3A4 enzyme induction by Compound 1 after repeated doses in Part 2.

Pharmacodynamic Parameters

Activity of RIPK1 when stimulated can be detected by an increase in autophosphorylation at serine 166 in the RIPK1 protein. Human PBMC are a primary cell type that express endogenous RIPK kinase. Therefore, the ability of Compound 1 to inhibit RIPK1 kinase activity, as measured by the inhibition of pS166-RIPK1 in prepared PBMC lysates isolated from blood samples, was investigated to assess the peripheral target engagement; pS166-RIPK1 levels in human PBMC lysates and percentage of change from baseline were calculated. In Part 1a study, six blood samples (at pre-dose, at 1, 3, 5, 8 hours on Day 1, and at 24 hours on Day 2) for pS166-RIPK1 level were collected per participant. In Part 2 study, blood samples were collected at timepoint (predose/baseline, D7 and D14 T0 (prior to dosing), at the followings timepoints at D14: T1h, T3H, T6H, T8h, then at D15/T24H and D16/T48H. Interquartile range (IQR: Q1-Q3) and geometric mean were added to the usual descriptive statistics.

A thorough time-matched (TM∘) ECG parameters analysis in relation with SAR443820 concentrations on Day 1 (SAD-Part 1a) and on Day 14 (MAD Part 2) over 24 hours compared to the baseline prior the first dosing was performed on ECG extracted from 24-hour Holter data.

Statistical Analyses

Safety population included all participants who received at least one dose of study intervention (Compound 1 or placebo). All safety assessments (AEs, laboratory parameters, vital signs) were based on the review of individual values and were summarized using descriptive statistics. Safety analysis was focused on treatment-emergent period, defined as the time from the first investigational medicinal product (IMP) administration up to the end of study visit (included). AEs were coded according to the Medical Dictionary for Regulatory Activities (MedDRA, version 23.1 in Part 1a and Part 1b; version 24 in Part 2). Severity was graded from Grade 1 to 4 according to the Food and Drug Administration (FDA) Guidance for Industry. Number (%) of participants experiencing TEAEs and potentially clinically significant abnormality (PCSAs) in clinical chemistry, vital signs, and ECGs were summarized by dose level group.

PK population included all participants exposed to Compound 1 with no major or critical deviations related to Compound 1 and for whom PK data were considered sufficient and interpretable. PK parameters were summarized by descriptive statistics for each dose level group. Distribution of Tₘₐₓ values was represented by histogram plots for each dose level group. In Part 1a, dose proportionality was assessed using a linear model on Cₘₐₓ, AUCₗₐₛₜ and AUC. In Part 2, all PK analyses were performed separately for each regimen (OD and BID). Steady state was assessed on C_{trough} using a non-linear model. Accumulation was assessed using a linear model on log-transformed accumulation ratio. Dose proportionality was assessed using a power model on Cₘₐₓ and AUCₜₐᵤ on Day 1 and Day 14, separately. Variance components of log-transformed Cₘₐₓ and AUCₜₐᵤ were estimated using a linear model. In Part 1b, CSF concentration was described by dose level group and CSF to plasma unbound concentration ratios calculated were described in the same way. 4β-hydroxycholesterol on Day 14 versus Day 1 pre-dose concentration ratios were summarized using descriptive statistics by dose level group and point estimates with 90% confidence interval (90% CI) for the geometric mean of the log-transformed Day14/Day1 ratio was calculated in Part 2.

Levels of pS166-RIPK1 and percentage of change from baseline were summarized descriptively by dose level group and time point. PD population includes participants included in the safety population with no major or critical deviations related to IMP and/or pharmacodynamic (PD) measurements, for whom the PD data are considered sufficient and interpretable will be included in the pharmacodynamic population. Scatterplots were provided for pS166-RIPK1 data versus PK plasma concentration in the PK/PD population. For each participant, the percent change compared to their baseline was evaluated at multiple timepoints up to 24 hours in Part 1a and up to 48 hours in Part 2.

For Part 1a (SAD) and Part 2 (MAD), the concentration-ECG population will encompass all randomized participants treated by SAR443820 as long as they have at least 1 ECG assessment time-matched with a pharmacokinetic concentration (ECGs centrally read coming from 24-hours Holter ECG). Participants randomized in the placebo group will also be included in the Concentration-ECG population if they have at least 1 non missing change from baseline in ECG parameter. Exposure-response analyses between QTcF parameters (change from time-matched baseline in 24-hours Holter ECG data) and corresponding Compound 1 concentrations were performed using graphical tools and regression methods over 24 hours on Day 1 (SAD-Part 1a) or Day 14 (MAD-Part 2) post-dose. Predictions at selected concentrations were computed using a linear mixed effect model (see Am Heart J. 2014; 168(3):262-72. J Pharmacokinet Pharmacodyn. 2018; 45(3):383-97).

Population Characteristics Analysis:

In Part 1a study, thirty-two healthy participants were administered one of the following single oral Compound 1 doses or the matched placebo capsules: 10, 20, 30 and 40 mg in fasted condition in cohorts of 8 participants each (6 Compound 1 and 2 placebo). All participants except one (lost to follow up for personal reason) completed the study.

All participants were between the ages of 19 and 55 years with a mean age of 31.8 years. Eleven (34.4%) participants were male and 21 (65.6%) were female. Twenty-five (78.1%) participants were White and 7 (21.9%) black or African American.

The safety and pharmacokinetic populations include 32 and 24 participants, respectively. The PD population includes 30 participants. Two participants in the 40 mg Compound 1 group were excluded from the PD analyses because the samples collected at baseline were incorrectly processed.

In Part 1b study, twelve healthy participants were administered one of the following single oral Compound 1 doses capsules: 10 and 40 mg in 2 cohorts of 6 participants each. All participants except one (declined further visits for follow-up) completed the study (Table 1).

All participants were between the ages of 19 and 54 years with a mean age of 29.4 years. Ten (83.3%) participants were male and 2 (16.7%) were female. Eleven (91.7%) participants were White and 1 (8.3%) black or African American. Both the safety and pharmacokinetic populations include 12 participants.

In Part 1b study, CSF concentration was described by dose level group and CSF to plasma unbound concentration ratios calculated were described in the same way. 40-hydroxycholesterol on Day 14 versus Day 1 pre-dose concentration ratios were summarized using descriptive statistics by dose level group and point estimates with 90% confidence interval (90% CI) for the geometric mean of the log-transformed Day14/Day 1 ratio was calculated in Part 2.

In Part 2 study, all participants included in this study (n=40) were between the ages of 19 and 55 years with a mean age of 32.1 years (±9.4). Twenty-five (62.5%) participants were male and 15 (37.5%) were female. Among 40 participants, 29 (72.5%) were Caucasian, 5 (12.5%) were Black or African American, 3 (7.5%) were Asian and 3 (7.5%) were of multiple origin. Baseline body weight ranged from 58.0 kg to 99.6 kg with a mean body weight of 79.19 (±10.49 kg) with no major difference between the dose level groups (Table 1).

TABLE 1
Demographics and baseline characteristics

| | Part 1a (SAD) | | | | | Part 1b | | | Compound 1 | | | |
|---|---|---|---|---|---|---|---|---|---|---|---|---|
| | | Compound 1 | | | | Compound 1 | | | 10 mg | 20 mg | 15 mg | 20 mg |
| | Placebo | 10 mg | 20 mg | 30 mg | 40 mg | 10 mg | 40 mg | Placebo | QD | QD | BID | BID |
| Characteristic | (N = 8) | (N = 6) | (N = 6) | (N = 6) | (N = 6) | (N = 8) | (N = 8) | (N = 8) | (N = 8) | (N = 8) | (N = 8) | (N = 8) |
| Age, years, | 34.5 | 29.0 | 36.2 | 32.8 | 25.3 | 29.3 | 29.5 | 35.0 | 31.0 | 28.9 | 37.5 | 28.3 |
| Mean (SD) | (11.0) | (5.9) | (12.8) | (10.7) | (8.2) | (11.9) | (12.9) | (13.2) | (5.1) | (8.1) | (10.8) | (6.4) |
| Sex, n (%), | 2 | 3 | 2 | 4 | 0 | 4 | 6 | 5 | 3 | 5 | 6 | 6 |
| Male | (25.0) | (50.0) | (33.3) | (66.7) | | (66.7) | (100.0) | (62.5) | (37.5) | (62.5) | (75.0) | (75.0) |
| Race, n (%), | 6 | 4 | 5 | 4 | 6 | 5 | 6 | 6 | 4 | 8 | 6 | 5 |
| White | (75.0) | (66.7) | (83.3) | (66.7) | (100.0) | (83.3) | (100.0) | (75.0) | (50.0) | (100) | (75.0) | (62.5) |
| Weight, kg, | 70.4 | 71.5 | 69.1 | 76.1 | 58.4 | 72.3 | 81.8 | 80.76 | 74.11 | 82.53 | 76.88 | 81.68 |
| Mean (SD) | (13.0) | (8.5) | (7.0) | (11.8) | (9.5) | (14.0) | (8.1) | (11.75) | (10.02) | (10.51) | (11.08) | (9.10) |
| BMI, Kg/m², | | | | | | | | | | | | |
| n (%) | | | | | | | | | | | | |
| 18.5 to <25 | 6 | 5 | 3 | 3 | 5 | 4 | 3 | 4 | 2 | 1 | 5 | 3 |
| | (75.0) | (83.3) | (50.0) | (50.0) | (83.3) | (66.7) | (50.0) | (50.0) | (25.0) | (12.5) | (62.5) | (37.5) |
| 25 to <30 | 2 | 1 | 3 | 3 | 1 | 2 | 3 | 4 | 5 | 6 | 3 | 5 |
| | (25.0) | (16.7) | (50.0) | (50.0) | (16.7) | (33.3) | (50.0) | (50.0) | (62.5) | (75.0) | (37.5) | (62.5) |
| ≥30 | 0 | 0 | 0 | 0 | 0 | 0 | 0 | 0 | 1 | 1 | 0 | 0 |
| | | | | | | | | | (12.5) | (12.5) | | |

Safety Results:

In Part 1a study, there were no SAEs or AESIs. One severe TEAE (blood creatinine phosphokinase increased) was observed in the placebo group. The highest frequency of participants with TEAEs was recorded for the nervous system disorders primary SOCs with 2 out of 6 (33.3%) participants in the 30 mg group, 2 out of 8 (25%) participants in the placebo group, and 1 out of 6 (16.7%) in each of the 20 mg and 40 mg group (Table 1). The most frequently reported TEAE was dizziness with 3 events in the 30 mg group, and 1 event each in the 40 mg group and the placebo group. Overall, fourteen (14) out of 32 participants experienced at least one TEAE: 5 out of 8 participants in the placebo group and 9 out 24 participants in Compound 1 groups, without any obvious dose-incidence relationship (Table 2, Table 3).

A few PCSAs scattered in the placebo, 10 mg and 20 mg Compound 1 groups were observed for hematology and clinical chemistry parameters. A few PC SA values were reported for vital sign parameters in the placebo, 10 mg, and 40 mg Compound 1 groups and for ECG parameters in all Compound 1 groups. All PCSAs in all Compound 1 groups were assessed as not clinically relevant.

In Part 1b study, there was 1 SAE (cerebrospinal fluid leakage) in Compound 1 40 mg group and 1 AESI (Alanine aminotransferase [ALT] increase) in Compound 1 10 mg group.

Cerebrospinal fluid leakage reported as AE of severe intensity occurred on Day 3 after 40 mg Compound 1 dosing and lumbar puncture performed as per protocol for CSF used for the measurement of Compound 1 concentration. The event was considered as serious because the participant's state required hospitalization. No other pathology was identified based upon investigations (MM). This SAE was a procedural complication of the lumbar puncture, considered as unrelated to Compound 1 40 mg. Corrective treatment consisted of blood patch applied on Day 5 leading to relief. This participant had radiculopathy and coccydynia of severe and moderate intensity, respectively on Day 3. Later (between Day 5 and Day 7), back pain and emesis, both of mild intensity were reported. All AEs were resolved.

ALT increase was reported on Day 2 after 10 mg Compound 1 dosing (1.5-fold ULN) and ALT levels increased to 2.4-fold ULN meeting the criterion of AESI on Day 7 (end-of-study visit). This AESI was considered as related to Compound 1 and not related to study procedure. All other liver tests (alkaline phosphatase, aspartate aminotransferase, total and direct bilirubin, and gamma glutamyl transferase) showed values within the normal ranges. No retest was performed since the participant declined further visits for follow-up. This isolated and asymptomatic ALT increase was not resolved.

Overall, ten (10) out of 12 participants experienced at least one TEAE: 4 out of 6 participants (66.7%) in Compound 1 10 mg group and all 6 participants in Compound 1 40 mg group, without any obvious dose-incidence relationship. Headache was the most frequently reported TEAE in this study, in 3 (50%) and 4 (83.3%) participants of the Compound 1 10 and 40 mg groups, respectively (Table 2, Table 3).

No PCSAs were observed for hematology and clinical chemistry parameters. A few PCSA values were reported for vital sign parameters and for ECG parameters in the Compound 1 10 mg and 40 mg groups. All PCSAs in all Compound 1 groups were considered as not clinically relevant.

In Part 2 study, there were no serious adverse event (SAE) and no severe treatment-emergent adverse event (TEAE, Grade ≥3) (Table 10). One adverse event of special interest (AESI) of alanine aminotransferase (ALT) increase occurred from Day 9 (2.29 ULN) in one participant in the Compound 1 15 mg BID group, and the highest ALT increased was observed on Day 15 (2.71 ULN, 24 hours post last dosing). This event was of Grade 1 severity, asymptomatic, not considered as serious, and did not lead to permanent study intervention discontinuation. The event was considered as related to the IMP by the Investigator.

Overall, 4 (50.0%), 3 (37.5%), 6 (75.0%), and 4 (50.0%) participants in the Compound 1 10 mg QD, 20 mg QD, 15 mg BID and 20 mg BID groups, respectively, and 5 (62.5%) participants in the placebo group experienced at least one TEAE during the study. The incidence of all TEAEs reported during the study was not imbalanced across the Compound 1 and the placebo groups.

The highest frequency of participants with TEAEs was recorded for the nervous system disorders primary system organ class with 2 out of 8 (25.0%) participants in each of the 10 mg QD and 20 mg BID groups and 1 out of 8 (12.5%) participants in each of the placebo, 20 mg QD and 15 mg BID groups. Headache was the most frequently reported TEAE, and its incidence was balanced across the Compound 1 and the placebo groups (Table 2).

There were few PCSAs observed in hematology and biochemistry parameters with no notable differences among the Compound 1 and placebo groups. Potentially clinically significant abnormalities for vital signs and ECG parameters were scattered in all groups and not clinically meaningful.

TABLE 2
Overall summary of TEAEs (safety population)

| | n (%) | | | | | | | | | | | |
|---|---|---|---|---|---|---|---|---|---|---|---|---|
| | | | | | | | | Part 2 | | | | |
| | Part 1a | | | | | Part 1b | | | Compound 1 | | | |
| | | Compound 1 | | | | Compound 1 | | | 10 mg | 20 mg | 15 mg | 20 mg |
| | Placebo | 10 mg | 20 mg | 30 mg | 40 mg | 10 mg | 40 mg | Placebo | QD | QD | BID | BID |
| | (N = 8) | (N = 6) | (N = 6) | (N = 6) | (N = 6) | (N = 8) | (N = 8) | (N = 8) | (N = 8) | (N = 8) | (N = 8) | (N = 8) |
| Any TEAE | 5 | 2 | 1 | 3 | 3 | 4 | 6 | 5 | 4 | 3 | 6 | 4 |
| | (62.5) | (33.3) | (16.7) | (50.0) | (50.0) | (66.7) | (100.0) | (62.5) | (50.0) | (37.5) | (75.0) | (50.0) |
| Any Grade ≥3 | 1 | 0 | 0 | 0 | 0 | 0 | 1 | 0 | 0 | 0 | 0 | 0 |
| TEAE | (12.5) | | | | | | (16.7) | | | | | |
| Any | 0 | 0 | 0 | 0 | 0 | 0 | 1 | 0 | 0 | 0 | 0 | 0 |
| treatment- | | | | | | | (16.7) | | | | | |
| emergent SAE | | | | | | | | | | | | |
| Any | 0 | 0 | 0 | 0 | 0 | 1 | 0 | 0 | 0 | 0 | 1 | 0 |
| treatment- | | | | | | (16.7) | | | | | (12.5) | |
| emergent AESI | | | | | | | | | | | | |

TABLE 3
Number (%) of participants with TEAE(s) by primary system organ class
and preferred term, by dose level group (safety population) in Part 1

| Primary system | Part 1a (SAD) | | | | | Part 1b | |
|---|---|---|---|---|---|---|---|
| organ class | | Compound 1 | | | | Compound 1 | |
| Preferred term n | Placebo | 10 mg | 20 mg | 30 mg | 40 mg | 10 mg | 40 mg |
| (%) | (N = 8) | (N = 6) | (N = 6) | (N = 6) | (N = 6) | (N = 6) | (N = 6) |
| Any class | 5 | 2 | 1 | 3 | 3 | 4 | 6 |
| | (62.5) | (33.3) | (16.7) | (50.0) | (50.0) | (66.7) | (100) |
| Nervous system | 2 | 0 | 1 | 2 | 1 | 4 | 5 |
| disorders | (25.0) | | (16.7) | (33.3) | (16.7) | (66.7) | (83.3) |
| Dizziness | 1 | 0 | 0 | 2 | 1 | 0 | 1 |
| | (12.5) | | | (33.3) | (16.7) | | (16.7) |
| Headache | 0 | 0 | 1 | 0 | 0 | 3 | 4 |
| | | | (16.7) | | | (50.0) | (66.7) |
| Presyncope | 1 | 0 | 0 | 0 | 0 | — | — |
| | (12.5) | | | | | | |
| Cerebrospinal | — | — | — | — | — | 1 | 3 (50.0) |
| fluid leakage | | | | | | (16.7) | |
| Radiculopathy | — | — | — | — | — | 0 | 1 |
| | | | | | | | (16.7) |
| Eye disorders | 0 | 0 | 0 | 1 | 0 | — | — |
| | | | | (16.7) | | | |
| Keratitis | 0 | 0 | 0 | 1 | 0 | — | — |
| | | | | (16.7) | | | |
| Cardiac disorders | 0 | 1 | 1 | 0 | 1 | — | — |
| | | (16.7) | (16.7) | | (16.7) | | |
| Ventricular | 0 | 1 | 0 | 0 | 1 | — | — |
| extrasystoles | | (16.7) | | | (16.7) | | |
| Tachycardia | 0 | 0 | 1 | 0 | 0 | — | — |
| | | | (16.7) | | | | |
| Vascular disorders | 0 | 0 | 0 | 1 | 0 | — | — |
| | | | | (16.7) | | | |
| Thrombophlebitis | 0 | 0 | 0 | 1 | 0 | — | — |
| | | | | (16.7) | | | |
| Skin and | 2 | 1 | 0 | 1 | 1 | — | — |
| subcutaneous | (25.0) | (16.7) | | (16.7) | (16.7) | | |
| tissue disorders | | | | | | | |
| Dermatitis | 1 | 1 | 0 | 1 | 1 | — | — |
| contact | (12.5) | (16.7) | | (16.7) | (16.7) | | |
| Skin irritation | 1 | 0 | 0 | 0 | 0 | — | — |
| | (12.5) | | | | | | |
| Gastrointestinal | — | — | — | — | — | 1 | 2 |
| disorders | | | | | | (16.7) | (33.3) |
| Constipation | — | — | — | — | — | 0 | 1 |
| | | | | | | | (16.7) |
| Nausea | — | — | — | — | — | 1 | 1 |
| | | | | | | (16.7) | (16.7) |
| Vomiting | — | — | — | — | — | 1 | 1 |
| | | | | | | (16.7) | (16.7) |
| Renal and urinary | — | — | — | — | — | 0 | 1 |
| disorders | | | | | | | (16.7) |
| Polyuria | — | — | — | — | — | 0 | 1 |
| | | | | | | | (16.7) |
| Musculoskeletal | 0 | 0 | 0 | 0 | 1 | 1 | 2 |
| and connective | | | | | (16.7) | (16.7) | (33.3) |
| tissue disorders | | | | | | | |
| Myalgia | 0 | 0 | 0 | 0 | 1 (16.7) | — | — |
| Back pain | — | — | — | — | — | 1 | 2 |
| | | | | | | (16.7) | (33.3) |
| Coccydynia | — | — | — | — | — | 0 | 1 |
| | | | | | | | (16.7) |
| General disorders | 1 | 0 | 0 | 1 | 1 | — | — |
| and administration | (12.5) | | | (16.7) | (16.7) | | |
| site conditions | | | | | | | |
| Vessel puncture | 0 | 0 | 0 | 0 | 1 | — | — |
| site | | | | | (16.7) | | |
| hypoaesthesia | | | | | | | |
| Vessel puncture | 0 | 0 | 0 | 0 | 1 | — | — |
| site pain | | | | | (16.7) | | |
| Fatigue | 0 | 0 | 0 | 1 | 0 | — | — |
| | | | | (16.7) | | | |
| Medical device | 1 | 0 | 0 | 0 | 0 | — | — |
| site dermatitis | (12.5) | | | | | | |
| Investigations | 1 | 0 | 0 | 0 | 0 | 1 | 0 |
| | (12.5) | | | | | (16.7) | |
| Blood creatine | 1 | 0 | 0 | 0 | 0 | — | — |
| phosphokinase | (12.5) | | | | | | |
| increased | | | | | | | |
| Alanine | — | — | — | — | — | 1 | 0 |
| aminotransferase | | | | | | (16.7) | |
| increased | | | | | | | |

Pharmacokinetic Results:

In Part 1a, PK parameters of Compound 1 following a single oral dose of 10 to 40 mg, in fasted conditions, and dose proportionality assessment are summarized in the tables below (Table 4, Table 5). In Part 1b, PK parameters of Compound 1 following a single oral dose of 10 or 40 mg, in fed state (Table 4), and CSF-to-unbound Plasma concentration ratio (Table 6) are summarized in the tables.

TABLE 4
Plasma pharmacokinetic parameters of SAR443820 (pharmacokinetic
population) in Part 1 of the study

| | | Part 1a | | | | Part 1b | |
|---|---|---|---|---|---|---|---|
| | | Compound 1 | | | | Compound 1 | |
| | | 10 mg | 20 mg | 30 mg | 40 mg | 10 mg | 40 mg |
| Parameter | Statistics | (N = 6) | (N = 6) | (N = 6) | (N = 6) | (N = 6) | (N = 6) |
| Cₘₐₓ (ng/ml) | Mean (SD) | 119 | 245 | 439 | 576 | 115 | 381 |
| | | (32.5) | (50.0) | (150) | (165) | (35.7) | (83.7) |
| | GM (% CV) | 116 | 241 | 417 | 555 | 111 | 374 |
| | | (27.2) | (20.5) | (34.2) | (28.7) | (31.0) | (22.0) |
| Tₘₐₓ (h) | Median | 1.02 | 1.50 | 1.00 | 1.50 | 4.24 | 4.50 |
| | (range) | (1.00, | (1.00, | (0.50, | (1.00, | (3.00, | (2.00, |
| | | 3.08) | 3.00) | 2.00) | 2.00) | 6.00) | 6.00) |
| AUCₗₐₛₜ | Mean (SD) | 1200 | 2400 | 2540 | 5390 | 1220 | 4100 |
| (h*ng/mL) | | (481) | (459) | (767) | (1860) | (447) | (1260) |
| | GM (% CV) | 11\20 | 2360 | 2420 | 5120 | 1170 | 3960 |
| | | (40.1) | (19.1) | (30.2) | (34.5) | (36.6) | (30.7) |
| AUC | Mean (SD) | 1250 | 2450 | 2570 | 5460 | 1460 | 5060 |
| (h*ng/mL) | | (510) | (466) | (775) | (1890) | (622) | (2150) |
| | GM (% CV) | 1170 | 2410 | 2440 | 5180 | 1370 | 4740 |
| | | (40.9) | (19.0) | (30.2) | (34.6) | (42.6) | (42.5) |
| Tₗₐₛₜ (h) | Median | 48.0 | 48.0 | 48.0 | 48.0 | 24.0 | 24.0 |
| | (range) | (24.0, | (24.0, | (24.0, | (24.0, | (24.0, | (24.0, |
| | | 48.1) | 48.0) | 48.0) | 48.0) | 24.0) | 24.0) |
| T_{1/2z} (h) | Mean (SD) | 8.22 | 7.63 | 5.99 | 6.40 | 8.09 | 8.31 |
| | | (2.39) | (1.63) | (1.91) | (1.32) | (1.57) | (2.45) |
| | GM (% CV) | 7.97 | 7.48 | 5.70 | 6.29 | 7.97 | 8.03 |
| | | (29.0) | (21.4) | (31.8) | (20.6) | (19.4) | (29.5) |
| V_{z}/F (L) | Mean (SD) | 100 | 90.2 | 102 | 71.7 | 86.0 | 98.6 |
| | | (20.2) | (12.8) | (12.3) | (17.0) | (22.0) | (14.1) |
| | GM (% CV) | 98.4 | 89.4 | 101 | 70.1 | 83.6 | 97.7 |
| | | (20.2) | (14.2) | (12.1) | (23.7) | (25.6) | (14.3) |
| CL/F (L/h) | Mean (SD) | 9.11 | 8.43 | 13.1 | 8.15 | 7.63 | 8.92 |
| | | (3.45) | (1.80) | (5.86) | (2.94) | (2.27) | (3.05) |
| | GM (% CV) | 8.56 | 8.28 | 12.3 | 7.72 | 7.27 | 8.43 |
| | | (37.8) | (21.4) | (44.7) | (36.1) | (29.7) | (34.2) |

In Part 1a, following single oral dose administration of 10 to 40 mg Compound 1 in fasted conditions, median tₘₐₓ was reached between 1.0 and 1.5 hours post dosing with no apparent dose effect. Following single oral dose administration of 10 and 40 mg Compound 1 in fed conditions (snack given less than 15 minutes before dosing), a delay is observed in tₘₐₓ with median values of 4.24- and 4.5-hours post-dose at 10 and 40 mg respectively as compared to 1.02- and 1.50-hours post-dose at 10 and 40 mg respectively in fasted state. The between-subject variability (CV %) of Compound 1 Cₘₐₓ, AUCs, and t_{1/2z} in fed conditions ranged from 19.4% to 42.6%.

The mean t_{1/2z} were similar across doses and ranged from 6 to 8 hours. The between-subject variability (CV %) of Compound 1 Cₘₐₓ, AUCs, and t_{1/2z} in fasted conditions ranged from 19% to 41%.

TABLE 5
Descriptive statistics of plasma pharmacokinetic parameters following single
oral administration of 10 to 40 mg Compound 1 in fasted conditions

| | | Point | |
|---|---|---|---|
| Parameter | Comparison | estimate | 90% CI |
| Cₘₐₓ | 40 mg vs. 30 mg (dose increase = 1.33) | 1.33 | (1.00 to 1.77) |
| | 30 mg vs. 20 mg (dose increase = 1.5) | 1.73 | (1.31 to 2.30) |
| | 20 mg vs. 10 mg (dose increase = 2) | 2.08 | (1.57 to 2.76) |
| | 40 mg vs. 20 mg (dose increase = 2) | 2.31 | (1.74 to 3.06) |
| | 30 mg vs. 10 mg (dose increase = 3) | 3.60 | (2.72 to 4.78) |
| | 40 mg vs. 10 mg (dose increase = 4) | 4.79 | (3.61 to 6.36) |
| AUCₗₐₛₜ | 40 mg vs. 30 mg (dose increase = 1.33) | 2.11 | (1.50 to 2.98) |
| | 30 mg vs. 20 mg (dose increase = 1.5) | 1.02 | (0.73 to 1.44) |
| | 20 mg vs. 10 mg (dose increase = 2) | 2.11 | (1.50 to 2.97) |
| | 40 mg vs. 20 mg (dose increase = 2) | 2.16 | (1.54 to 3.05) |
| | 30 mg vs. 10 mg (dose increase = 3) | 2.16 | (1.53 to 3.04) |
| | 40 mg vs. 10 mg (dose increase = 4) | 4.56 | (3.24 to 6.42) |
| AUC | 40 mg vs. 30 mg (dose increase = 1.33) | 2.12 | (1.51 to 2.98) |
| | 30 mg vs. 20 mg (dose increase = 1.5) | 1.01 | (0.72 to 1.42) |
| | 20 mg vs. 10 mg (dose increase = 2) | 2.07 | (1.47 to 2.90) |
| | 40 mg vs. 20 mg (dose increase = 2) | 2.15 | (1.53 to 3.01) |
| | 30 mg vs. 10 mg (dose increase = 3) | 2.09 | (1.49 to 2.94) |
| | 40 mg vs. 10 mg (dose increase = 4) | 4.43 | (3.16 to 6.23) |

Compound 1 exposures increased with dose with no major deviation from dose proportionality between 10 and 40 mg.

Regarding Cₘₐₓ, a 2-fold increase in dose such as 10 to 20 mg led to a 2.08-fold (90% CI: 1.57 to 2.76) or from 20 to 40 mg led to a 2.31-fold increase (90% CI: 1.74 to 3.06).

For AUC and AUClast, a 2-fold increase in dose, 10 to 20 mg or 20 to 40 mg led to a 2.07-fold (90% CI:1.47 to 2.90) or a 2.15-fold increase (90% CI: 1.53 to 3.01) for AUC and a 2.11-fold (90% CI:1.50 to 2.97) or a 2.16-fold increase (90% CI: 1.54 to 3.05) for AUClast.

TABLE 6
CSF-to-unbound Plasma concentration ratio

| Parameter | Statistics | [T1H-T3H] | [T4H-T6H] | [T5H-T16H] |
|---|---|---|---|---|
| CSF-to- | N | 4 | 4 | 3 |
| unbound Plasma | Mean | 0.840 | 0.804 | 1.271 |
| concentration | Geom Menu | 0.817 | 0.755 | 1.266 |
| ratio | SD | 0.240 | 0.306 | 0.135 |
| | CV | 29% | 38% | 11% |
| | Median | 0.732 | 0.825 | 1.307 |
| | Minimum | 0.70 | 0.44 | 1.12 |
| | Maximum | 1.20 | 1.13 | 1.38 |

In the time intervals [T1H-T3H] or [T4H-T6H] the ratio CSF to unbound plasma was about 0.8 while in the time interval [T8H-T10H] it was around 1.3.

Pharmacokinetic Results for Part 2 Study:

Pharmacokinetic parameters after single and 14-day oral doses of Compound 1 administered in healthy participants are summarized in Table 7 and Table 8, respectively.

TABLE 7
Descriptive statistics of plasma pharmacokinetic parameters
following a first oral Compound 1 dose administration in
fasted condition by dose level and dosing regimen

| | Parameter | | Treatment Description | | | |
|---|---|---|---|---|---|---|
| Day | (Unit) | | 10 mg QD | 20 mg QD | 15 mg BID | 20 mg BID |
| 1 | Cₘₐₓ | N | 8.00 | 8.00 | 8.00 | 8.00 |
| | (ng/mL) | Mean ± SD | 161 ± 33.2 | 245 ± 49.5 | 229 ± 101 | 209 ± 42.4 |
| | | (Gmean) | (157) [20.7] | (240) [20.2] | (211) [44.2] | (205) [20.3] |
| | | [CV] | | | | |
| 1 | Tₘₐₓ (h) | N | 8.00 | 8.00 | 8.00 | 8.00 |
| | | Median [Min-Max] | 1.00 | 1.50 | 1.00 | 2.00 |
| | | | [0.500-2.00] | [1.00-2.00] | [0.500-2.00] | [1.00-4.00] |
| 1 | AUCₜₐᵤ | N | 8.00 | 8.00 | 8.00 | 8.00 |
| | (h*ng/ml) | Mean ± SD | 1110 ± 268 | 1910 ± 429 | 1150 ± 380 | 1370 ± 252 |
| | | (Gmean) | (1080) | (1860) [22.5 | (1100) [33.1] | (1350) |
| | | [CV] | [24.2] | | | [18.4] |

The dosing interval tau is 12 hours for the BID cohorts and 24 hours for the QD cohorts.

TABLE 8
Descriptive statistics of plasma pharmacokinetic parameters at steady state
following Compound 1 14-days repeated oral administration in fasted condition by dose level and
dosing

| | Parameter | | Treatment Description | | | |
|---|---|---|---|---|---|---|
| Day | (Unit) | | 10 mg QD | 20 mg QD | 15 mg BID | 20 mg BID |
| 14 | Cₘₐₓ | N | 7.00 | 8.00 | 8.00 | 7.00 |
| | (ng/mL) | Mean ± SD | 164 ± 52.3 | 255 ± 58.0 | 284 ± 103 | 359 ± 71.6 |
| | | (Gmean) | (158) [31.9] | (250) [22.7] | (268) [36.3] | (353) [19.9] |
| | | [CV] | | | | |
| 14 | Tₘₐₓ (h) | N | 7.00 | 8.00 | 8.00 | 7.00 |
| | | Median | 1.00 | 2.00 | 1.00 | 1.00 |
| | | [Min-Max] | [1.00-2.00] | [0.600-2.00] | [1.00-2.00] | [0.500-2.00] |
| 14 | AUCₜₐᵤ | N | 7.00 | 8.00 | 8.00 | 7.00 |
| | (h*ng/mL) | Mean ± SD | 1460 ± 399 | 2170 ± 465 | 1710 ± 556 | 2220 ± 574 |
| | | (Gmean) | (1410) [27.4] | (2130) [21.4] | (1640) [32.5] | (2160) [25.9] |
| | | [CV] | | | | |
| 14 | T_{1/2z} (h) | N | 7.00 | 8.00 | 8.00 | 7.00 |
| | | Mean ± SD | 9.07 ± 2.11 | 8.68 ± 1.65 | 7.29 ± 1.14 | 7.54 ± 1.74 |
| | | (Gmean) | (8.87) [23.2] | (8.53) [19.0] | (7.21) [15.6] | (7.37) [23.0] |
| | | [CV] | | | | |
| 14 | CL_{SS}/F | N | 7.00 | 8.00 | 8.00 | 7.00 |
| | (L/h) | Mean ± SD | 7.30 ± 1.89 | 9.60 ± 2.14 | 9.54 ± 2.86 | 9.53 ± 2.37 |
| | | (Gmean) | (7.08) [25.9] | (9.40) [22.2] | (9.16) [30.0] | (9.27) [24.9] |
| | | [CV] | | | | |
| 14 | C_{trough} | N | 7.00 | 8.00 | 8.00 | 7.00 |
| | (ng/mL) | Mean ± SD | 22.8 ± 13.0 | 34.2 ± 16.6 | 83.7 ± 27.4 | 107 ± 42.7 |
| | | (Gmean) | (19.9) [57.0] | (30.4) [49.0] | (79.3) [33.0] | (99.3) [40.0] |
| | | [CV] | | | | |

The dosing interval tau is 12 hours for the BID cohorts and 24 hours for the OD cohorts.

The median steady state of Compound 1 plasma concentrations was reached within about 1.4-2 days after first administration irrespective of the dose or the dosing regimen. After single or repeated administration of Compound 1 in fasted conditions, median Tₘₐₓ ranged between 1.00-2.00 hours post-dose. At Day 1, for the BID dosing regimen, Cₘₐₓ did not increase between 15 mg and 20 mg. At steady state, Cₘₐₓ and AUCₜₐᵤ increased with the dose both for the QD and BID regimen, T_{1/2z} remained similar across doses and dosing regimen and mean values ranged between 7.3-9.1 hours.

With 2-fold increase in dose from 10 to 20 mg QD, Cₘₐₓ and AUCₜₐᵤ increased by 1.58 (90% CI: 1.26-1.99) and 1.51-fold (90% CI: 1.21-1.88) at Day 14, respectively. With 1.33-fold increase in dose from 15 to 20 mg BID, Cₘₐₓ and AUCₜₐᵤ increased by 1.31 (90% CI: 1.00-1.73) and 1.32-fold (90% CI: 1.01-1.72), respectively at Day 14.

The steady state achievement based on C_{trough} using a non-linear model is summarized in Table 9 and Table 10 for QD and BID dosing regimen, respectively.

TABLE 9
Estimate of median and 90th percentile for Compound 1 Tₛₛ for
cohorts at 10 and 20 mg QD
Time to steady state (days)

| Dose | Quantile | Point estimate | 90% CI* |
|---|---|---|---|
| Overall | 90% | 2.45 | (2.10 to 2.47) |
| | 50% Median | 1.81 | (1.63 to 2.10) |

*Distribution free confidence intervals

TABLE 10
Estimate of median and 90th percentile for Compound 1 Tss
for cohorts at 15 and 20 mg BID
Time to steady state (days)

| Dase | Quantile | Point estimate | 00% CI* |
|---|---|---|---|
| Overall | 90% | 1.65 | (1.46 to 2.05) |
| | 50% Median | 1.43 | (1.32 to 1.46) |

*Distribution free confidence intervals
Note:
Due to OD dose regimen at D14, D14 12H was considered in place of D15 T0H as last C_{trough} for the steady state calculation.
Day 1 T12H was also taken into consideration in this steady state calculation

The dose proportionality assessment at Day 14 is summarized in Table 11 and Table 12 for the QD and BID cohorts, respectively.

TABLE 11
Dose proportionality assessment for Compound 1 Cₘₐₓ and AUCₜₐᵤ
following 14-day repeated oral administration (Day 14)
for cohorts at 10 and 20 mg QD

| | | Ratio | |
|---|---|---|---|
| Parameter | Dose ratio | Point estimate | 90% CI |
| Cₘₐₓ | (r) = 2 | 1.58 | (1.26 to 1.99) |
| | Beta Estimate | 0.66 | (0.33 to 0.99) |
| AUC₀₋₂₄ₕ | (r) = 2 | 1.51 | (1.21 to 1.88) |
| | Beta Estimate | 0.59 | (0.27 to 0.91) |

Cmax = 34.33 × dose ^{0.66}
AUC0-24h = 361.82 × dose ^{0.59}
Note:
Due to a premature treatment discontinuation one subject (10 mg QD) has no PK data on Day 14.

TABLE 12
Dose proportionality assessment for Compound 1 Cₘₐₓ and
AUCₜₐᵤ following 14-day repeated oral administration
(Day 14) for cohorts at 15 and 20 mg BID

| | | Ratio | |
|---|---|---|---|
| Parameter | Dose ratio | Point estimate | 90% CI |
| Cₘₐₓ | (r) = 1.3333 | 1.31 | (1.00 to 1.73) |
| | Beta Estimate | 0.95 | (−0.00 to 1.90) |
| AUC₀₋₁₂ₕ | (r) = 1.3333 | 1.32 | (1.01 to 1.72) |
| | Beta Estimate | 0.96 | (0.04 to 1.88) |

Cₘₐₓ = 20.43 × dose ^{0.95}
AUC₀₋₁₂ₕ = 122.37 × dose ^{0.96}
Note:
Due to a premature treatment discontinuation one subject (20 mg BID) has no PK data on Day 14.

The accumulation ratios assessments and the variance component are summarized in are summarized in Table 13 and Table 14 for QD cohorts, in Table 15 and Table 16 for BID cohorts.

TABLE 13
Accumulation ratio on Cₘₐₓ and AUCₜₐᵤ following 14-day repeated
oral administration of Compound 1 for QD cohorts

| Parameter | Study intervention group | Point estimate | 90% CI |
|---|---|---|---|
| Ratio | Compound 1 10 mg QD | 1.00 | (0.86 to 1.17) |
| (Cₘₐₓ) | Compound 1 20 mg QD | 1.06 | (0.91 to 1.22) |
| | Pooled Compound 1 | 1.03 | (0.93 to 1.14) |
| | doses QD | | |
| Ratio | Compound 1 10 mg QD | 1.31 | (1.22 to 1.41) |
| (AUC₀₋₂₄ₕ) | Compound 1 20 mg QD | 1.13 | (1.05 to 1.21) |
| | Pooled Compound 1 | 1.22 | (1.16 to 1.28) |
| | doses QD | | |

TABLE 14
Variance components on Cₘₐₓ and AUCₜₐᵤ for QD cohorts

| | Within-subject SD | | Total SD | |
|---|---|---|---|---|
| | Point | | Point | |
| Parameter | estimate | 90% CI | estimate | 90% CO |
| Log(Cₘₐₓ) | 0.158 | (0.121 to 0.235) | 0.206 | (0.169 to 0.286) |
| Log(_{AUC0-24h}) | 0.080 | (0.061 to 0.118) | 0.216 | (0.168 to 0.315) |

TABLE 15
Accumulation ratio on Cₘₐₓ and AUCₜₐᵤ following 14-day repeated oral
administration of Compound 1 for BID cohorts

| Parameter | Study intervention group | Point estimate | 90% CI |
|---|---|---|---|
| Ratio | Compound 1 15 mg BID | 1.24 | (1.04 to 1.48) |
| (Cₘₐₓ) | Compound 1 20 mg BID | 1.71 | (1.42 to 2.05) |
| | Pooled Compound 1 doses BID | 1.46 | (1.27 to 1.67) |
| Ratio | Compound 1 15 mg BID | 1.45 | (1.30 to 1.61) |
| (AUC₀₋₁₂ₕ) | Compound 1 20 mg BID | 1.63 | (1.46 to 1.82) |
| | Pooled Compound 1 doses BID | 1.54 | (1.42 to 1.66) |

Note:
Due to a premature treatment discontinuation one subject (20 mg BID) has no accumulation ratio (Day 14/Day 1).

TABLE 16
Variance components on Cₘₐₓ and AUCₜₐᵤ for BID cohorts

| | Within-subject SD | | Total SD | |
|---|---|---|---|---|
| | Point | | Point | |
| Parameter | estimate | 90% CI | estimate | 90% CI |
| Log(Cₘₐₓ) | 0.179 | (0.136 to 0.265) | 0.322 | (0.257 to 0.461) |
| Log(AUC₀₋₁₂ₕ) | 0.113 | (0.086 to 0.168) | 0.284 | (0.222 to 0.413) |

The accumulation ratio for Cmax and AUCtau appeared similar between doses in QD and BID cohorts. The within-subject variability for Cmax and AUCtau was estimated at 15.8% and 8.0% for QD cohorts, and 17.9% and 11.3% for BID cohorts, respectively.

Pharmacodynamics Results:

The amount of phosphorylation at serine 166 in RIPK1 protein (pS166-RIPK1) was measured in PBMC lysates prepared from blood samples. Variability in baseline values between individuals was observed across the treatment groups. For each participant, the percent change compared to their baseline was evaluated at multiple timepoints. A negative value for percent change indicates an inhibition of phosphorylation of RIPK1 at-serine166 (pS166-RIPK1). Inhibition of the phosphorylation at this site was observed in all the Compound 1 dose groups reflecting a target engagement of Compound 1 with RIPK1 doses.

In Part 1a study, median inhibition greater than 80% was observed in all Compound 1 dose groups (10 mg, 20 mg, 30 mg and 40 mg) at as early as 1 hour post dose and continued to exceed this level in all up to 5 hours post dose. In contrast, no apparent level of inhibition was detected in the placebo group (of note, 2 participants of the 40 mg Compound 1 were excluded). The highest median inhibition was observed in the 20 mg Compound 1 group ranging from 90.43% at 1 hr post dose and peaking to 96.63% at the 5 hrs timepoint, maintaining greater than 94% median inhibition over 24 hours. Peak median inhibition for the 30 mg cohort was achieved at 3 hr and represented a median inhibition of 91.48%, decreasing to a median of 80.16% inhibition at 24 hours post dose but the bioanalyses were conducted on only one replicate. Likewise, the peak median inhibition of the 40 mg Compound 1 group with PD data obtained from 4 participants was achieved at 1 hr post dose with a median of 86.69%.

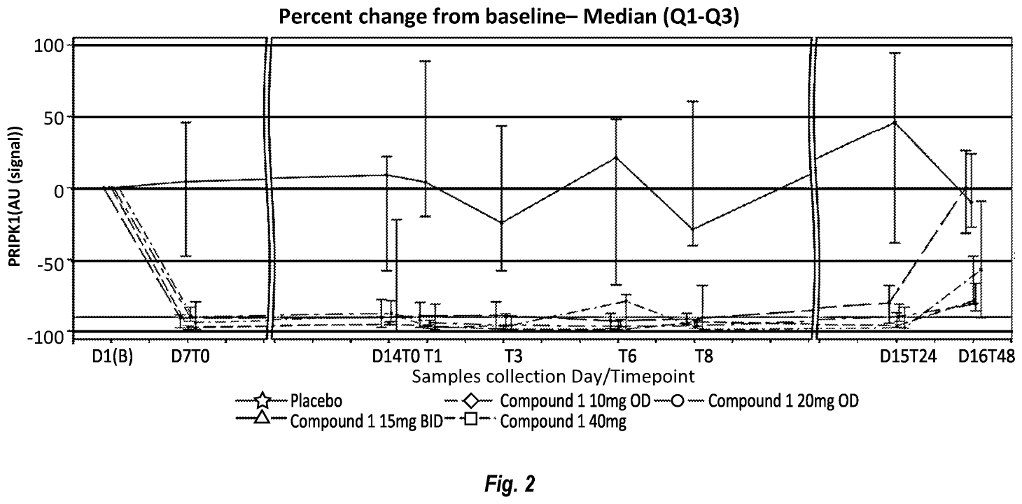

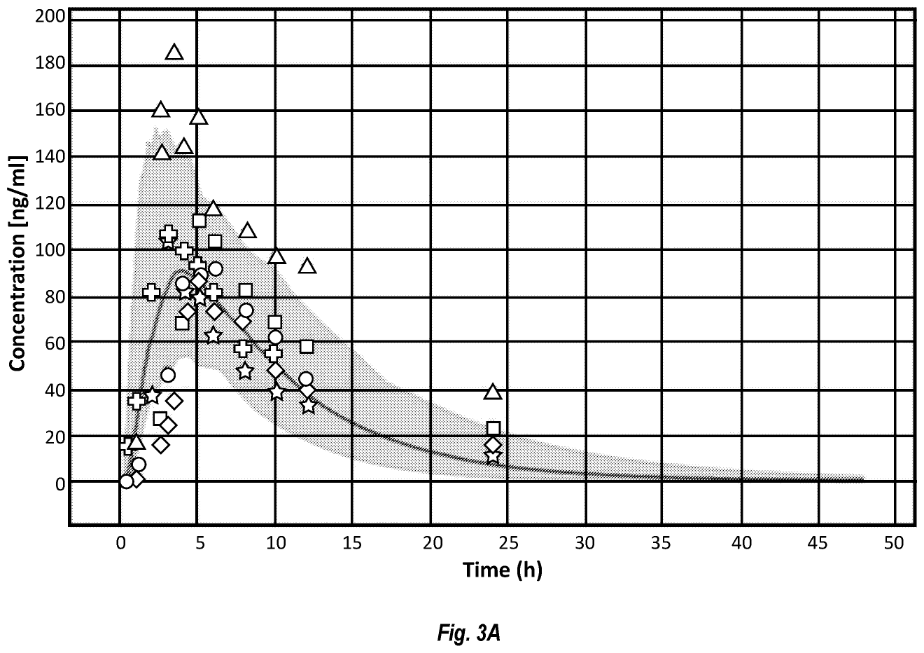

In Part 2 study, inhibition of the phosphorylation at Serine 166-RIPK1 was observed in all the Compound 1 dose level groups (from 10 mg QD to 20 mg BID) close to 90% at C_{trough} Day 7 (first post-baseline timepoint) and up to 24 hours post-last dosing, reflecting target engagement of Compound 1 with RIPK1 (FIG. 2).

On Day 14, a maximal effect was observed 6 hours post-last morning dose with a median inhibition of 99.05% (10 mg QD group, n=5), 96.53% (20 mg QD group, n=4), 92.80% (15 mg BID group, n=7), and at 8 hours post-last morning dose with a median inhibition of 99.19% (20 mg BID group, n=3). The inhibition was maintained up to 24 hours post-last morning dosing at Day 14 (Day15-T24h) with a range from −80.29% (15 mg BID group) to −97.76% (20 mg BID group).

At 48 hours post-last dosing, the inhibition became reduced in all dose level groups. Variability in the basal level of pS166-RIPK levels was observed but no apparent level of inhibition was detected in the placebo group. While small differences were observed in overall levels of RIPK1 inhibition, there was no apparent difference between the dose level groups.

Effect of Compound 1 Repeated Doses on CYP3A4 Induction

The statistical analysis of 40-hydroxycholesterol data was conducted on the PK population (N=32 participants). Point-estimates and 90% CIs of the ratio Day 14/Day 1 were provided by Compound 1 dose level group along with the pooled Compound 1 dose level groups using an ANCOVA model performed on individual log-transformed Day 14/Day 1 ratios.

For 1 participant from Compound 1 20 mg QD group, the concentration dosed at Day 1 was not kept in the statistical analysis because the sample tube was hemolyzed. Moreover, the ratio Day 14/Day 1 was not calculated for the 2 participants prematurely withdrawn before Day 14 (1 participant in the Compound 1 10 mg QD group and 1 participant in the Compound 1 20 mg BID group). The ratio Day 14/Day 1 was not calculated for these 3 participants. Point estimates with 90% CIs of the ratio of 40-hydroxycholesterol plasma concentrations calculated from samples collected on Day 14 and on Day 1 (pre-dose) are presented in Table 17 and Table 18.

Point estimates of Day 14/Day 1 4β-hydroxycholesterol ratio were 0.88 (90% CI: 0.82 to 0.95) and 1.02 (90% CI: 0.91 to 1.14) for pooled QD and pooled BID dosing regimen, respectively, suggesting that there is no potential for CYP3A4 induction by Compound 1 after repeated doses (up to 20 mg BID) in healthy adult participants. 4b-hydroxycholesterol levels after 14 days of multiple QD or BID doses did not increase (vs baseline value), suggesting no potential for CYP3A4 induction by repeated Compound 1 doses (up to 20 mg BID).

TABLE 17
Day 14/Day 1 ratio of 4-b hydroxycholesterol-plasma
concentration-Compound 1 administered in QD regimen-Point
estimates of 4-b hydroxycholesterol with 90% confidence interval

| | | Point | |
|---|---|---|---|
| Parameter | Group | estimate | 90% CI |
| Ratio(4-β- | SAR443820 10 mg OD | 0.80 | (0.72 to 0.89) |
| hydroxycholesterol) | SAR443820 20 mg OD | 0.98 | (0.88 to 1.10) |
| | SAR443820 OD pooled | 0.88 | (0.82 to 0.95) |

Day 14/Day I ratio of 4-β hydroxycholesterol was assessed for each dose level group separately as well as pooled across dose level groups

TABLE 18
Day 14/Day 1 ratio of 4-b hydroxycholesterol-plasma
concentration-Compound 1 administered in BID regimen-Point
estimates of 4-b hydroxycholesterol with 90% confidence interval

| | | Point | |
|---|---|---|---|
| Parameter | Group | estimate | 90% CI |
| Ratio(4-β- | SAR443820 15 mg BID | 1.07 | (0.93 to 1.24) |
| hydroxycholesterol) | SAR443820 20 mg BID | 0.97 | (0.84 to 1.13) |
| | SAR443820 BID pooled | 1.02 | (0.91 to 1.14) |

Day 1|4/Day 1 ratio of 4-β hydroxycholesterol was assessed for each dose level group separately as well as pooled across dose level groups

Concentration-QTcF Analysis

No clinically relevant dose-related effects of Compound 1 on QTcF intervals were observed, indication no potential for Compound 1, when administered at single dose up to 40 mg in or at repeated dose up to 20 mg BID, to cause QTcF modification (Table 19).

TABLE 19
Predicted values and 90% CIs for delta QTcF, HR, PR and QRS vs placebo at
selected concentrations (concentration-ECG population) - Day 1 and Day 14

| | Part 1a (SAD) | | | | Part 2 (MAD) | | | | |
|---|---|---|---|---|---|---|---|---|---|
| ECG | | Cₘₐₓ | Predicted value | | | | Cₘₐₓ | Predicted value | |
| parameter | Compound 1 | (ng/ml)^{a} | (msec) (90% CI) | | Compound 1 | | (ng/ml)^{a} | (msec) (90% CI) | |
| QTcF | 10 mg | 116.00 | −4.41 | (−11.32, 2.50) | 10 | mg QD | 158.00 | −5.36 | (−13.23, 2.50) |
| Interval | 20 mg | 241.00 | −3.88 | (−10.68, 2.91) | 20 | mg QD | 250.00 | −4.01 | (−12.06, 4.04) |
| (msec) | 30 mg | 417.00 | −3.15 | (−9.96, 3.66) | 15 | mg BID | 268.00 | −3.74 | (−11.85, 4.36) |
| | 40 mg | 555.00 | −2.57 | (−9.55, 4.40) | 20 | mg BID | 353.00 | −2.49 | (−10.97, 5.98) |
| Heart Rate | 10 mg | 116.00 | −3.68 | (−7.51, 0.15) | 10 | mg QD | 158.00 | 0.25 | (−3.84, 4.34) |
| (beats/min) | 20 mg | 241.00 | −2.62 | (−6.45, 1.22) | 20 | mg QD | 250.00 | −0.18 | (−4.42, 4.07) |
| | 30 mg | 417.00 | −1.11 | (−5.16, 2.93) | 15 | mg BID | 268.00 | −0.26 | (−4.56, 4.04) |
| | 40 mg | 555.00 | 0.06 | (−4.32, 4.45) | 20 | mg BID | 353.00 | −0.65 | (−5.27, 3.96) |
| PR Interval | 10 mg | 116.00 | 5.87 | (−0.52, 12.25) | 10 | mg QD | 158.00 | −4.63 | (−15.69, 6.44) |
| (msec) | 20 mg | 241.00 | 6.70 | (0.29, 13.12) | 20 | mg QD | 250.00 | −4.55 | (−15.63, 6.54) |
| | 30 mg | 417.00 | 7.88 | (1.21, 14.54) | 15 | mg BID | 268.00 | −4.53 | (−15.63, 6.57) |
| | 40 mg | 555.00 | 8.80 | (1.75, 15.85) | 20 | mg BID | 353.00 | −4.46 | (−15.69, 6.77) |
| QRS | 10 mg | 116.00 | −0.04 | (−3.15, 3.07) | 10 | mg QD | 158.00 | 2.52 | (−0.76, 5.81) |
| Duration | 20 mg | 241.00 | 0.43 | (−2.68, 3.54) | 20 | mg QD | 250.00 | 2.92 | (−0.52, 6.36) |
| (msec) | 30 mg | 417.00 | 1.10 | (−2.14, 4.34) | 15 | mg BID | 268.00 | 3.00 | (−0.49, 6.48) |
| | 40 mg | 555.00 | 1.62 | (−1.84, 5.09) | 20 | mg BID | 353.00 | 3.37 | (−0.37, 7.10) |

### CONCLUSIONS

Compound 1 was safe and well tolerated up to a single dose of 40 mg in fasted (SAD-Part 1a) and fed (Part 1b) conditions and up to the highest dose of 20 mg BID administered in fasted conditions for 14 days in healthy male and female participants.

There were no SAEs, severe TEAEs or TEAEs leading to permanent treatment discontinuation reported. In Part 2 study, one participant experienced an AESI of asymptomatic ALT increase of mild intensity (Grade 1) which did not lead to study discontinuation and resolved. There were few treatment-emergent PCSAs for the laboratory evaluations, vital signs, and ECG parameters, none of them were considered clinically relevant.

In Part 1a study, the dose escalation was stopped at 40 mg due to human exposure limits (i.e., Cmax of 3.0 μM and an AUC of 20 μM*h, which correspond to approximately 1/10 of no-observed-adverse-effect-level (NOAEL)-Cmax and NOAEL-AUC, respectively, in the 3-month study in monkey).

In Part 1a study, following single oral dose administration of 10 to 40 mg Compound 1 in fasted conditions, median tmax was reached between 1.0 and 1.5 hours post dosing with no apparent dose effect. Compound 1 exposures increased according to dose proportionality between 10 and 40 mg. The mean estimate of t½z across all doses ranged from 6 to 8 hours. The between-subject variability (CV %) of Compound 1 Cmax, AUCs, and t½z in fasted conditions ranged from 19% to 41%.

In Part 2 study, after repeated administration of Compound 1 in fasted condition, median Tₘₐₓ ranged from 1 to 2 h across dose levels. The mean elimination half-lives appeared similar across doses and ranged from 7.3 to 9.1 h on Day 14.

In Part 2 study, median steady state of Compound 1 plasma concentrations was reached within of 1.4 to 2 days after first administration whatever the dose or the dosing regimen.

In Part 2 study, data observed on Day 14 after 10 mg and 20 mg QD showed that Compound 1 exposures increased less than expected by dose proportionality with an increase in Cₘₐₓ of 1.58-fold and in AUCₜₐᵤ of 1.51-fold, for a 2-fold increase in dose. After repeated dosing of Compound 1, almost no or limited accumulation was observed, as ratios between Days 14 and 1 were around 1.03 for Cₘₐₓ and 1.22 for AUCₜₐᵤ for the QD dosing cohorts.

Data observed on Day 14 after 15 mg and 20 mg BID showed that Compound 1 exposure increased with no major deviation from dose proportionality with a 1.31-fold increase for Cₘₐₓ and a 1.32-fold increase in AUCₜₐᵤ, for a 1.33-fold increase in dose. After repeated dosing an overall accumulation around 1.5 was observed for both Cₘₐₓ and AUCₜₐᵤ in the BID dosing cohorts.

Total variability was 20.6% and 32.2% for Cₘₐₓ and 21.6% and 28.4% for AUCₜₐᵤ, for the QD and the BID dosing regimen respectively. The within-subject variability was 15.8% and 17.9% for Cₘₐₓ and 8.0% and 11.3% for AUCₜₐᵤ, for the QD and BID dosing regimen, respectively. Compound 1 exhibits low within-subject PK variability and low to moderate total variability.

In Part 1a study, a marked inhibition between 82% and 97% as measured by change from baseline for pS166-RIPK1 levels was observed in all Compound 1 treated groups reflecting a target engagement of Compound 1 with RIPK1 at all single doses administered in the SAD part from 10 to 40 mg, and not observed in the placebo group. In Part 2 study, inhibition of the pS166-RIPK1 was observed in all the Compound 1 dose level groups (from 10 mg QD to 20 mg BID) close to 90% at C_{trough} Day 7 (first post-baseline timepoint) and up to 24 hours post-last dosing, reflecting high target engagement of Compound 1 with RIPK1.

### Example 2: Exploratory Population Pharmacokinetic/Pharmacodynamic (popPK/PD) Analysis

An exploratory population pharmacokinetic/pharmacodynamic analysis of phosphorylated receptor-interacting protein kinase 1(pRIPK1) in healthy participants after single (SAD) and repeated (MAD) administrations of Compound 1 was carried out to develop a population PK/PD model from data after single and repeated doses in healthy adults and to derive individual parameters for each dose cohort.

The population PK/PD analysis was performed using the stochastic approximation expectation maximization (SAEM) algorithm for nonlinear mixed-effects models implemented in MONOLIX software (R1 2020). The observed plasma concentrations were used to develop the model with plasma concentrations from placebo participants and blow limit of quantitation (BLQ) plasma concentrations from verum participants being fixed to zero. Classical direct and indirect inhibitory models were evaluated. Model selection was based on objective function value Akaike and Bayesian Information criteria as well as standard goodness-of-fit plots (Psychol Methods. 2012 June; 17(2):228-43; Pharmaceutics. 2020 June; 12(6): 578.). From selected final population PK/PD model, individual PD parameters were derived and summarized by descriptive statistical analyses by study and dose level.

A total of 433 samples in 70 participants was considered (101, 121 and 211 samples after placebo, single dose and repeated doses, respectively). No data from potential outliers were excluded. In placebo participants, a very large inter-cohort and inter-individual variability was observed. In verum participants, a deep and reversible inhibition of pRIPK1 was observed in all cohorts, after both single and repeated administrations. Selection of structural PK/PD model confirmed that a direct relationship best described the data overall. From individual observed PD vs PK data plotted by increasing sampling times, only few participants showed some hysteresis profiles. Population PK/PD model parameters are presented in Table 20. Typical Iₘₐₓ and IC₅₀ values were 95.1% and 1.68 ng/mL respectively confirming the high efficacy and potency of Compound 1 on pRIPK1 inhibition.

$$PK / PD model equation Effect = E 0 * ( 1 - \frac{I max * {c}^{γ}}{IC {50}^{γ} + {c}^{γ}} )$$

TABLE 20
Population PK/PD model parameters

| Parameter | Estimate | RSE (%) |
|---|---|---|
| Imax (%) | 95.1 | 2.08 |
| IC50 (ng/ml) | 1.68 | 23.8 |
| E0 (AU_signal) | 8541 | 9.94 |
| Gamma | 1.17 | 26.3 |
| ω(Imax) (%) | 192 | 13.4 |
| ω(IC50) (%) | 71.4 | 25.4 |
| ω(E0) (%) | 73.7 | 11.7 |
| 0(Gamma) (%) | 49.5 | 54.9 |
| σprop (%) | 44.5 | 4.41 |

Abbreviations: E0: Baseline effect; IC50: concentration needed for 50% of Imax; Imax: maximum inhibitory effect; RSE: Relative Standard Error

Typical EC50, EC90 and EC95 (plasma concentration needed to get 50, 90 and 95% decrease in pharmacodynamic effect vs baseline) are indicated in the table below (Table 21).

TABLE 21
Derived typical EC50, EC90 and EC95

| | Plasma conc (ng/mL) |
|---|---|
| EC50-50% decrease in E0 | 1.83 |
| EC90-90% decrease in E0 | 19.7 |
| EC91-91% decrease in E0 | 23.9 |
| EC92-92% decrease in E0 | 30.7 |
| EC93-93% decrease in E0 | 43.3 |
| EC94-94% decrease in E0 | 76.1 |
| EC95-95% decrease in E0 | 600 |

Abbreviations: E0: Baseline effect; ECxx: concentration necessary to get xx % decrease in E0

After single and repeated doses in healthy adults, a direct sigmoidal Imax model best described the relationship between observed Compound 1 plasma concentrations and observed levels of pRIPK1. Typical values for maximal inhibition (Iₘₐₓ) and plasma concentration needed to get I_{max/2} pharmacodynamic effect (IC₅₀) were 95.1% and 1.68 ng/mL, respectively. All tested dose levels in Example 1 and Example 2 showed a deep, almost complete and reversible inhibition of pRIPK1 over the time-course after dosing.

### Example 3: Physiologically-Based Pharmacokinetic (PBPK) Study

A preliminary whole-body physiologically based pharmacokinetic model (PBPK) was developed using in vitro data and pharmacokinetic data in preclinical species. The model was further refined with results from Example 1 and Example 2. The Compound 1 concentrations obtained in plasma in Part 1A (10, 20, 30 mg) were used for model calibration, while data from part 1A (40 mg) and data in plasma from Part 1B (10, 40 mg) was used as an external validation for the model. After the model validation in the prediction of the full PK profiles for 10 and 40 mg, the model was used to predict the CSF concentrations after repeated dosing.

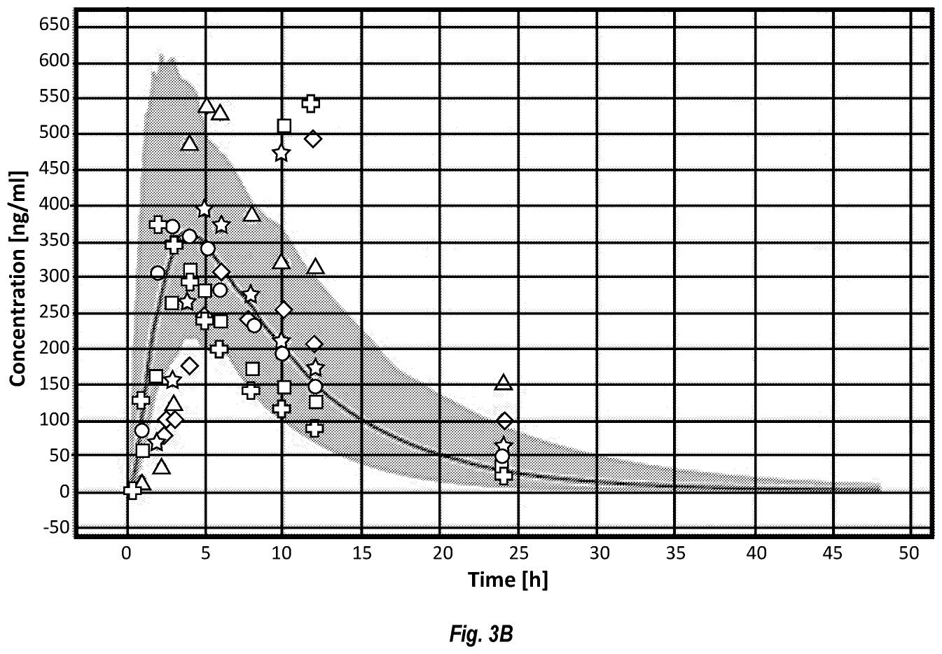

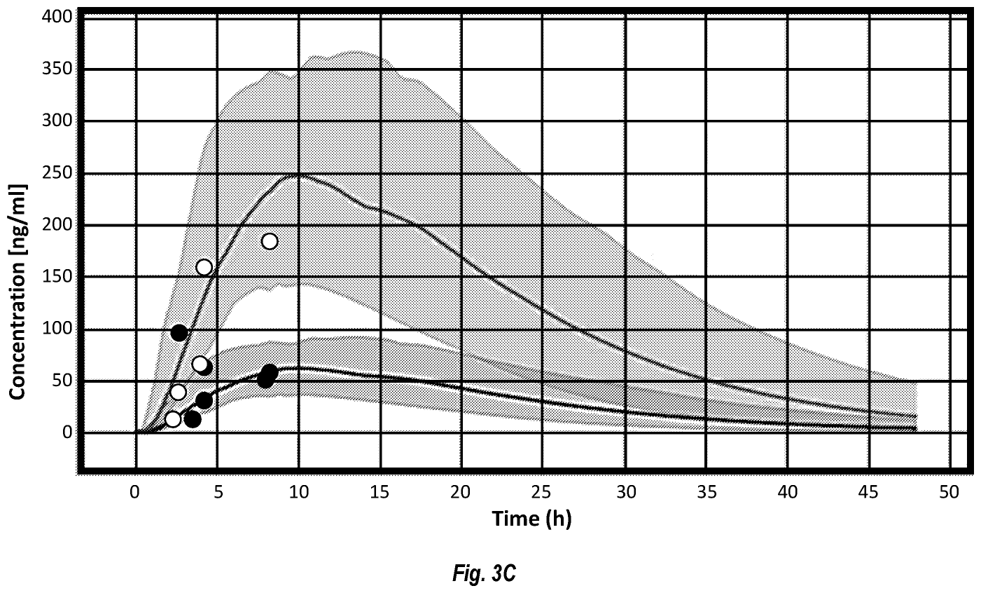

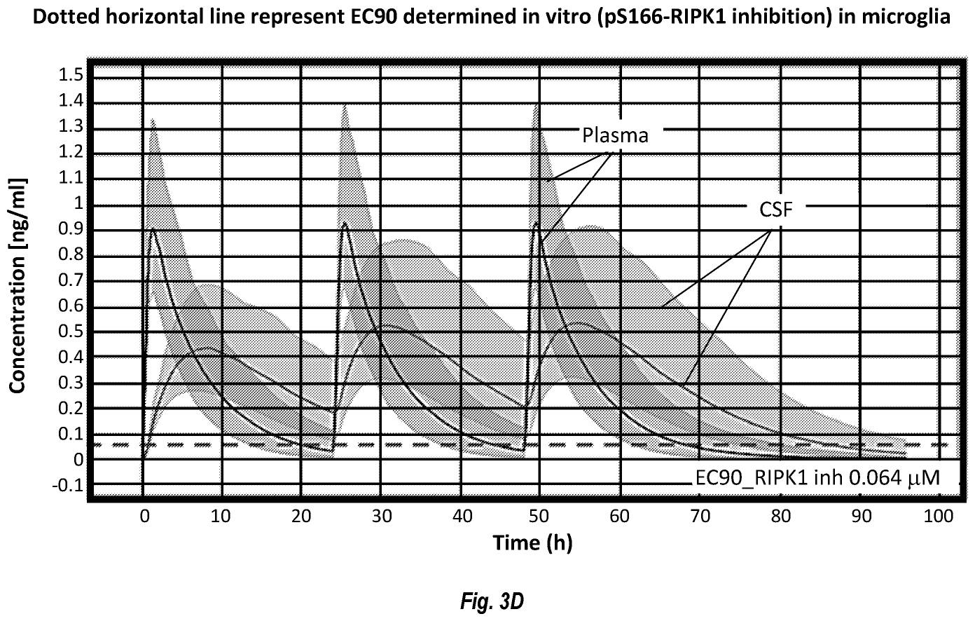

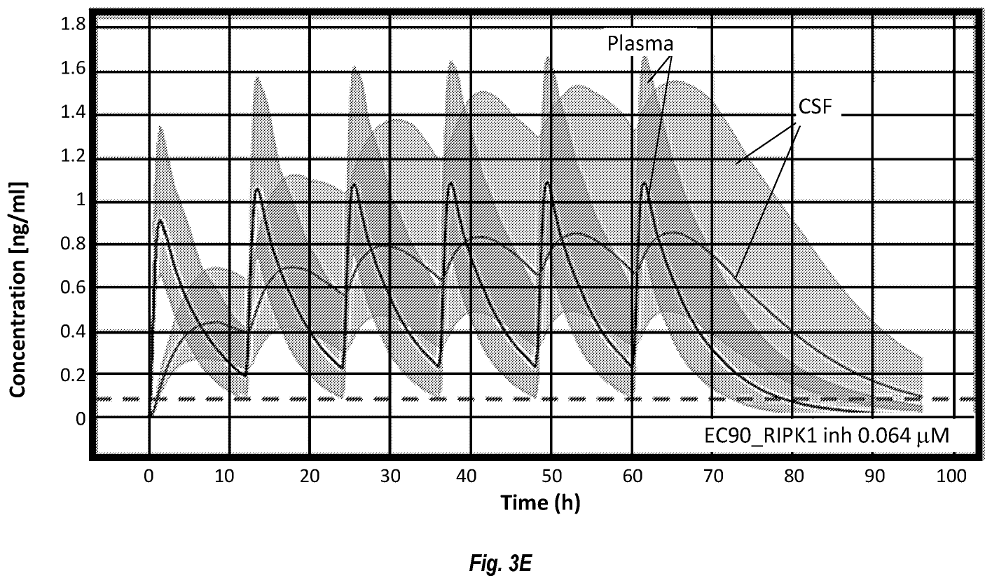

The predicted Compound 1 concentration in CSF after repeated administration at 20 mg QD and 20 mg BID are expected to be above EC₉₀ (pS166-RIPK1 inhibition) determined either in vitro in microglia (0.064 μM) or in vivo in PBMC (0.038 μM), during the dosing interval, indicating an appropriate exposure at central level for targeting high level of target engagement (FIG. 3A-3E).

### Example 4: Dose Regimen Examples

### Example 4-1: 20 mg BID

The dose of 20 mg BID Compound 1 is estimated to provide a high level of target engagement required for obtaining a clinical effect.

In the first-in-human study (see Examples 1 and 2), Compound 1 up to a highest single dose of 40 mg and multiple 14-day dose of 20 mg BID, was found to be safe and well tolerated. In the Phase 1a SAD study, there were no SAEs. All the TEAEs were of mild to moderate intensity except 1 severe TEAE (blood creatine phosphokinase [CPK] increase of Grade 3) reported in the placebo group. Since convulsions were assumed to be related to Cₘₐₓ based on scientific rationale and the fact that it occurred close to the tₘₐₓ in the nonhuman primate study, a BID dose was tested in the MAD study to reduce Cₘₐₓ while maintaining an equivalent AUC to that of the highest single dose of 40 mg and reaching a higher C_{trough}. In the MAD study, no SAEs, other than dose limiting AEs or laboratory findings, were reported. All the AEs reported in the MAD study were of mild to moderate intensity.

At 20 mg BID, the highest dose administered, the mean observed Cₘₐₓ is 1.22 μM which is <10-fold lower compared to Cₘₐₓ of 13.1 μM at the NOAEL in female rats at the end of the Week 26 6-month study; and the AUC₀₋₂₄ h (corresponding to AUC₀₋₁₂ h*2) was 15.0 μM*hour which is <8-fold lower compared to the AUC₀₋₂₄ of 126 μM*h at the NOAEL in female rats at end of Week 26 study in the 6-month study and which is <10-fold margin based on the exposure at the male NOAEL.

The dose of 20 mg BID Compound 1 is estimated to provide a high level of target engagement required for obtaining a clinical effect. Preliminary data indicate a good penetration of Compound 1 in CSF with mean values of the CSF/unbound plasma ratio close to 1. Based on a preliminary PBPK model, the predicted SAR443820 concentration in CSF after repeated administration at 20 mg BID is expected to be above IC90 (pS166-RIPK1 level determined either in vitro in microglia or in vivo in PBMC), during the dosing interval, indicating an appropriate exposure at central level for achieving high level of target engagement. Therefore, 20 mg BID, which has been demonstrated to be well tolerated in healthy subjects, is estimated to have good target engagement while maintaining a 10-fold safety margin relative to the NOAEL exposure in the nonhuman primate study.

### Example 4-2: 20 mg QD

The dose of 20 mg QD Compound 1 has previously demonstrated strong target engagement and was well tolerated in healthy participants.

In the FIH study, Compound 1 up to a highest single dose of 40 mg and 14-day dose of Compound 1 20 mg BID, was found to be safe and well tolerated. In the Phase 1a SAD study, there were no SAEs. All the TEAEs were of mild to moderate intensity except 1 severe TEAE (blood creatine phosphokinase [CPK] increase of Grade 3) reported in the placebo group. In the MAD study, no SAEs were reported. All the AEs reported in the MAD study were of mild to moderate intensity. At 20 mg QD, the mean observed Cₘₐₓ and AUC₀₋₂₄ at steady state are 0.86 μM and 7.35 μM*hour, respectively based on MAD study that are 15 and 17-fold lower, respectively than the exposure at NOAEL in the 6-month rat toxicology study.

A preliminary PK-PD model has been developed using PD data (pS166-RIPK measurement in human PBMCs) from SAD and MAD studies and indicates high efficacy (Iₘₐₓ=95%) and potency (IC₅₀=1.68 ng/mL) of Compound 1 on pRIPK1 inhibition. Good penetration of Compound 1 in CSF has been observed in human with mean values of the CSF/unbound plasma ratio close to 1.0. The preliminary PBPK model indicates that at a dose of 20 mg QD, the predicted SAR443820 concentration in CSF is expected to be above IC90 (pS166-RIPK1 level determined either in vitro in microglia or in vivo in PBMC), during the dosing interval, indicating an appropriate exposure at central level for achieving high level of target engagement. The dose of 20 mg QD Compound 1 is estimated to provide a high level of target engagement required for obtaining a clinical effect.

### Example 4-3: 15 mg BID

The dose of 15 mg BID Compound 1 has previously demonstrated strong target engagement and was well tolerated in healthy participants.

In the FIH study, Compound 1 up to a highest single dose of 40 mg and 14-day dose of Compound 1 20 mg BID, was found to be safe and well tolerated. In the Phase 1a SAD study, there were no SAEs. All the TEAEs were of mild to moderate intensity except 1 severe TEAE (blood creatine phosphokinase [CPK] increase of Grade 3) reported in the placebo group. In the MAD study, no SAEs were reported. All the AEs reported in the MAD study were of mild to moderate intensity. At 15 mg BID, the mean observed Cₘₐₓ and AUCₜₐᵤ at steady state are 284 ng/mL and 1710 h*ng/mL, which are 10-fold lower than the exposure at NOAEL in the 6-month rat toxicology study.

The preliminary PK-PD model indicates high efficacy (Iₘₐₓ=95%) and potency (IC₅₀=1.68 ng/mL) of Compound 1 on pRIPK1 inhibition. Good penetration of Compound 1 in CSF has been observed in human with mean values of the CSF/unbound plasma ratio close to 1.0. The preliminary PBPK model indicates that at a dose of 15 mg BID, the predicted SAR443820 concentration in CSF after repeated administration at 20 mg BID is expected to be above IC90 (pS166-RIPK1 level determined either in vitro in microglia or in vivo in PBMC), during the dosing interval, indicating an appropriate exposure at central level for achieving high level of target engagement. The dose of 15 mg BID Compound 1 is estimated to provide a high level of target engagement required for obtaining a clinical effect.

### Example 5: A Phase 2, Multicenter, Randomized, Double-Blind, Placebo-Controlled Study to Evaluate the Efficacy and Safety of Compound 1 in Adult Participants with Amyotrophic Lateral Sclerosis, Followed by an Open-Label Extension

This is a parallel-treatment, Phase 2, randomized, double-blind study to assess the efficacy, safety, tolerability, PK, and PD of 20 mg twice daily (BID) oral Compound 1 compared with placebo in male and female participants aged 18 to 80 years with ALS followed by an open-label, long-term extension period.

The study will consist of 2 parts: Part A of the study will last for 24 weeks, and participants will receive 20 mg BID oral Compound 1 or placebo in a double-blind fashion for 24 weeks. All ongoing participants at Week 24 will rollover to open-label extension (Part B.). Part B begins at the end of Week 24 and continues up to Week 106.

The primary endpoints are: 1) Change from baseline in the ALSFRS-R total score—Part A; 2) Combined assessment of the function and survival (CAFS) score—Part B. Secondary endpoints are: 1) Combined assessment of the function and survival (CAFS) score—Part A; 2) Change from baseline in slow vital capacity (SVC)—Part A; 3) Muscle Strength—Part A; 4) Change from baseline in Amyotrophic Lateral Sclerosis Assessment Questionnaire (ALSAQ-5)—Part A; 5) Change from baseline in serum neurofilament light chain (NfL) —Part A; 6) Number of patients with treatment-emergent adverse events (TEAE) and Serious adverse event (SAE)—Part A; 7) Assessment of pharmacokinetic parameter—Plasma concentration of SAR443820-Part A; 8) Combined assessment of the function and survival (CAFS) score—Part B; 9) Change from baseline in the ALSFRS R total score-Part B; 10) Time from baseline to the occurrence of either death, or permanent assisted ventilation (>22 hours daily for >7 consecutive days), whichever comes first—Part B; 11) Time from baseline to the occurrence of death—Part B; 12) Change from baseline in slow vital capacity (SVC)-Part B; 13) Change from baseline in Amyotrophic Lateral Sclerosis Assessment Questionnaire (ALSAQ-5)-Part B; 14) Change from baseline in serum neurofilament light chain (NfL)-Part B; 15) Number of patients with treatment emergent adverse events (TEAE) and Serious adverse event (SAE)—Part B; 16) Assessment of pharmacokinetic parameter Plasma concentration of SAR443820-Part B.

Some patients will be excluded from this study: 1) Exclusion of participants with history of seizures or epilepsy (except a history of febrile seizure during childhood). 2) Exclusion of participants at risk of higher exposure (participants weighing <45 kg, or participants with moderate or severe hepatic impairment). 3) Not allowing concomitant medications that may increase the exposure, such as potent and moderate cytochrome P450 (CYP)3A4 inhibitors. 4) Convulsions and seizures are identified as adverse events of special interest (AESI) to ensure reporting within 24 hours to the Sponsor's Global Pharmacovigilance Database. If the seriousness criteria are met, convulsions are reported as SAEs. Subsequently per process, Convulsions are considered Adverse Events of Special Interest (AESIs) which will be monitored closely, and reported diligently and rapidly, per process, to the FDA. 5) If a convulsion is identified in any single participant, the participant will discontinue the administration of investigational medicinal product (IMP) immediately and permanently, no matter the cause of convulsions.

Participants should be prohibited from consuming grapefruit or grapefruit juice (including pomelos, exotic citrus fruits, grapefruit hybrids, or fruit juices) from 5 days before the start of the study intervention until 2 days after the final dose.

1. A method of treating a receptor-interacting protein kinase 1-mediated disease or disorder, comprising administering to a subject in need thereof a therapeutically effective amount of 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof.

2. The method of claim 1, wherein the 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof is administered at a dose of about 20 mg.

3. The method of claim 1, wherein the 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof is administered at a dose of about 20 mg QD.

4. The method of claim 1, wherein the 4-(3,3-difluoro-2,2-dimethyl-propanoyl)-3,5-dihydro-2H-pyrido[3,4-f] [1,4]oxazepine-9-carbonitrile carbonitrile or a pharmaceutically acceptable salt thereof is administered at a dose of about 20 mg BID.

5. The method of claim 2, wherein the dose is administered once daily.

6. The method of claim 2, wherein the dose is administered twice daily.

7. The method of claim 2, wherein the dose is administered with food.

8. The method of claim 2, wherein the dose is administered without food.

9. The method of claim 2, wherein the dose is administered for 24 weeks.

10. The method of claim 1, wherein the 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof, is administered as monotherapy.

11. The method of claim 1, wherein the subject is a human.

12. (canceled)

13. The method of claim 1, wherein the receptor-interacting protein kinase 1-mediated disease or disorder is selected from at least one of Niemann-Pick disease type C1 (NPC1), amyotrophic lateral sclerosis (ALS), Friedreich's ataxia, Huntington's disease, Lewy body disease, multiple sclerosis, Parkinson's disease, multiple sclerosis, and spinal muscular atrophy.

14. A method of treating a receptor-interacting protein kinase 1-mediated disease or disorder, comprising administering to a subject in need thereof a therapeutically effective amount of 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof at a dose of about 20 mg QD.

15. The method of claim 14, wherein the receptor-interacting protein kinase 1-mediated disease or disorder is selected from at least one of Niemann-Pick disease type C1 (NPC1), amyotrophic lateral sclerosis (ALS), Friedreich's ataxia, Huntington's disease, Lewy body disease, multiple sclerosis, Parkinson's disease, multiple sclerosis, and spinal muscular atrophy.

16. A method of treating a receptor-interacting protein kinase 1-mediated disease or disorder, comprising administering to a subject in need thereof a therapeutically effective amount of 4-(3,3-difluoro-2,2-dimethyl-propanoyl)-3,5-dihydro-2H-pyrido[3,4-f] [1,4]oxazepine-9-carbonitrile or a pharmaceutically acceptable salt thereof at a dose of about 20 mg BID.

17. The method of claim 16, wherein the receptor-interacting protein kinase 1-mediated disease or disorder is selected from at least one of Niemann-Pick disease type C1 (NPC1), amyotrophic lateral sclerosis (ALS), Friedreich's ataxia, Huntington's disease, Lewy body disease, multiple sclerosis, Parkinson's disease, multiple sclerosis, and spinal muscular atrophy.
